SEQUENCE LISTING (8 sequences)

SEQ ID NO: 1 - DNA, 1182 bp, Homo sapiens
gcggccggcggcgtctcctcccgggacgctgaggggcccgaggagaccgtgaggctctggcctgcagctcgcgccgccatggacgctgccgaggtcgaattcctcgccgagaaggagctggttaccattatccccaacttcagtctggacaagatctacctcatcgggggggacctggggccttttaaccctggtttacccgtggaagtgcccctgtggctggcgattaacctgaaacaaagacagaaatgtcgcctgctccctccagagtggatggatgtagaaaagttggagaagatgagggatcatgaacgaaaggaagaaacttttaccccaatgcccagcccttactacatggaacttacgaagctcctgttaaatcatgcttcagacaacatcccgaaggcagacgaaatccggaccctggtcaaggatatgtgggacactcgtatagccaaactccgagtgtctgctgacagctttgtgagacagcaggaggcacatgccaagctggataacttgaccttgatggagatcaacaccagcgggactttcctcacacaagcgctcaaccacatgtacaaactccgcacgaacctccagcctctggagagtactcagtctcaggacttctagagaaaggcctggtgcaggcggcttgctgggggatgtgagcgctcaggatgtgatgaggtactcgtggttctggagctctagaaacacttctgatgcatgaaaaatgtgtgatggtgcaaggaatggattcaggatgttgttggagaaacaagtttgtgattagtccttaaaacttagctccctgggacattcttcaattccacatctgtttctagaaaccagccctttttccccccacttttgagaaataaaaaagccttaggtaaataagtcattctccctagcagagccacttgggtctcctgcatggaagccgtcacacttgggcaggtgttcagtgactggtaggtgtagatacagcaggagtggccatgtggtccacggctttttaccccttcttgatcctgatttcttgggctgaatttagactctctcacagaggtggctcacagagaaggatggcagatggtgcagccaacaatgctgaccggtgcttatcctctaagccctgatccacaataaaaatggacccaactcaaaaaaaaa

SEQ ID NO: 2 - protein, 185 aa, Homo sapiens
MDAAEVEFLAEKELVTIIPNFSLDKIYLIGGDLGPFNPGLPVEVPLWLAINLKQRQKCRLLPPEWMDVEKLEKMRDHERKEETFTPMPSPYYMELTKLLLNHASDNIPKADEIRTLVKDMWDTRIAKLRVSADSFVRQQEAHAKLDNLTLMEINTSGTFLTQALNHMYKLRTNLQPLESTQSQDF

SEQ ID NO: 3 - DNA, 21 bp, Artificial Sequence
  Sense RNA oligonucleotide specific for bsh3.
cccugguuuacccguggaatt

SEQ ID NO: 4 - DNA, 21 bp, Artificial Sequence
  Antisense RNA oligonucleotide for bsh3.
uuccacggguaaaccagggtt

SEQ ID NO: 5 - DNA, 552 bp, Schizosaccharomyces pombe
atggccttaccaagggaacttgaaatttcattctcaccagaggaaatggaatttttagctggcaatgagtatattaacattgttccttcagaaacaatggatcaattgccgctagttagcgcgacgattcccattatgaagcctcccaaaaaatgtcgggttcctttatggttggctcttgagctaaaaaagcaaaatttggcaaggattgtacctccggaatggatggaaataggcaaacttgaaaacatacgagatgatgaattggaaaacgagacattctcagagttgccttttcggtggctagaaacggcgcatttattgttaaacttctgtgctgatgatatcgaggatgttgaggatatcaggaggattttgctggacattcgagaagctagacaaagtaaagcaagaacgggattggaagccattaatgaagttcaactaactttggataatttgggtgcaatggaaattaacgaaatacgtcctatatttcgagaggtgatggacagaatgcgcaaaattgttcaagtttcccaagaagaatga

SEQ ID NO: 6 - protein, 183 aa, Schizosaccharomyces pombe
MALPRELEISFSPEEMEFLAGNEYINIVPSETMDQLPLVSATIPIMKPPKKCRVPLWLALELKKQNLARIVPPEWMEIGKLENIRDDELENETFSELPFRWLETAHLLLNFCADDIEDVEDIRRILLDIREARQSKARTGLEAINEVQLTLDNLGAMEINEIRPIFREVMDRMRKIVQVSQEE

SEQ ID NO: 7 - DNA, 42 bp, Artificial Sequence
  Forward primer.
cgttgtggatccgacgctgccgaggtcgaattcctcgccgag

SEQ ID NO: 8 - DNA, 42 bp, Artificial Sequence
  Reverse primer.
ctctagtctagactagaagtcctgagactgagtactctccag

(19) United States
(12) Patent Application Publication
(10) Pub. No.: US 2006/0057153 A1
(43) Pub. Date: Mar. 16, 2006

(54) Method and composition for the inhibition of mitosis

(76) Inventors: Tony Hunter, Del Mar, CA (US); Han-Kuei Huang, San Diego, CA (US)

Correspondence Address: KLARQUIST SPARKMAN, LLP, 121 S.W. SALMON STREET, SUITE 1600, PORTLAND, OR, 97204, US

(21) Appl. No.: 10/463,059

(22) Filed: Jun. 16, 2003

Related U.S. Application Data
(60) Provisional application No. 60/389,686, filed on Jun. 17, 2002.

(51) Int. Cl. C12Q 1/68; G01N 33/574; A61K 39/00; A61K 48/00; A61K 31/4745

(52) U.S. Cl. 424/185.1; 514/44; 514/283; 435/6; 435/7.23

(57) ABSTRACT

A function for the human protein, Bsh3 was identified herein as being involved in mitosis. Thus, inhibitors of Bsh3 can be used for the treatment of cancer or other diseases marked by over-proliferation. Inhibitors of the yeast homolog (S. pombe Bsh3) can be used as anti-fungals. Alternatively, Bsh3 or Bsh3 activators may be used to activate proliferation in cells such as tissue culture cells and transplants.

This application claims priority of U.S. Provisional application 60/389,686, filed Jun. 17, 2002, herein incorporated by reference in its entirety.

### GOVERNMENTAL INTEREST

Certain aspects of the invention disclosed herein were made with United States government support under National Institutes of Health grant CA-80100. The United States government may have certain rights in these aspects of the invention.

### FIELD OF THE INVENTION

The present invention relates generally to methods and compositions for the inhibition of cell growth and mitosis. More specifically, the invention relates to the use of Bsh3 protein and its homologues, and compounds that interact with Bsh3 and its homologues, to affect cell growth and mitosis.

### BACKGROUND OF THE INVENTION

A hallmark feature of cancerous cells is uncontrolled proliferation, due in part to uncontrolled mitosis. Among the differences that have been discovered between tumor and normal cells are resistance to the process of programmed cell death, also known as apoptosis, as well as aberrantly increased cellular mitosis.

Many proteins involved in the process of mitosis have also been found to be associated with the uncontrolled proliferation of cancer cells. For example, overexpression of Survivin, a recently described member of the IAP (inhibitor of apoptosis) caspase inhibitor family has been detected in tumors of the lung, colon, pancreas, prostate, breast, stomach, non Hodgkin's lymphoma, and neuroblastoma. A number of Survivin homologs have now been identified which were all found to be involved in mitotis and to be essential for survival. Such homologs of Survivin include the C. elegans homolog, BIR-1, the S. cerevisiae homolog, Bir1p, and the S. pombe homolog, Bir1p/Cut17p/Pbh1p, which is a nuclear protein required for viability, chromosome segregation and cytokinesis.

Although Survivin appears to be essential, it is likely that there are a variety of proteins which may act in parallel, in addition to, and instead of Survivin in regulation of cellular mitosis. These proteins are likely to play a role in the uncontrolled proliferation seen with cancer cells. Thus, the identification of proteins involved in mitosis may lead to further treatments and diagnostics for cancer.

### SUMMARY OF THE INVENTION

Embodiments of the invention relate to the use of Bsh3, and its homologs, to affect cellular mitosis. In one embodiment, the homologue is the Bsh3p protein from S. pombe or the Bsh3 homologue from humans. More specifically, Bsh3 appears to play a role in chromosome segregation during cell growth.

One embodiment of the invention is the use of Bsh3 as an agent, or as a target for agents, that inhibit or stimulate Bsh3 -mediated activation of cellular mitosis. For example, inhibitors of Bsh3 are useful to block abnormal cell growth. In addition, activators of Bsh3 are useful to extend cell growth.

Another embodiment is a method for identifying a compound that modulates Bsh3 levels in a cell, including contacting the cell with a test compound and monitoring the level of Bsh3 in the cell. A change in the level of mitosis of cells contacted with a compound relative to the level of mitosis in cells not contacted with a compound is indicative of a compound that modulates mitosis. The compound can be an activator, or an inhibitor, of Bsh3 expression or activity.

A further embodiment includes methods for the identification of compounds that act as inhibitors of Bsh3 activity or expression, including compounds that inhibit the activity or expression of proteins or molecules that bind to Bsh3 or are located downstream of directly Bsh3 binding proteins or molecules. Inhibitors may be identified by any method known to one of skill in the art. Inhibitors include, but are not limited to, small molecules, antibodies, nucleic acids, peptides and proteins.

A further embodiment is methods for identifying compounds that act as activators of Bsh3 activity or expression, including compounds that inhibit the activity or expression of molecules that bind to Bsh3, or are located downstream of Bsh3-binding molecules. Activators may be identified by any method known to one of skill in the art. Activators include, but are not limited to, small molecules, antibodies, nucleic acids, peptides and proteins.

A further embodiment includes methods for isolating proteins that bind to Bsh3. Probes based on the Bsh3 protein or fragments of Bsh3, as discussed below, are used as capture probes to isolate Bsh3 binding proteins. Dominant negative proteins, DNAs encoding these proteins, antisense inhibitors of these proteins, antibodies to these binding proteins, peptide fragments of these proteins or mimics of these proteins may be introduced into cells to affect Bsh3 function.

Additionally, Bsh3 or its binding partners provide novel targets for screening of synthetic small molecules and combinatorial or naturally occurring compound libraries to discover novel therapeutics to regulate Bsh3 function.

Still another embodiment of the invention is a method for increasing the number of cells in vitro by contacting cells with an effective amount of Bsh3, or an activator of Bsh3 activity, or expression. For example, such cells may be stem cells or cells that are to be transplanted.

Yet another embodiment of the invention is a method for reducing the number of cancer or tumor cells in an individual by contacting the cancer or tumor cells with an effective amount of a inhibitor of Bsh3. Alternatively, the cancer or tumor cells can be contacted with an anti-Bsh3 compound, or an active variant thereof.

A further embodiment of the invention is a method for reducing the number of autoimmune reactive cells in an individual by contacting the reactive cells with an effective amount of an anti-Bsh3 compound or an active variant thereof.

One other embodiment is a method for reducing the number of fugal cells in an individual or a plant by contacting the fungal cells with an effective amount of an anti-Bsh3 compound or an active variant thereof.

An additional embodiment is a method of monitoring the proliferation potential of a population of cells by determining the level of Bsh3 activity or expression in the cell population. A high level of Bsh3 activity or expression is indicative of high proliferation potential. A low level of Bsh3 activity or expression is indicative of low proliferation potential.

Another embodiment is a method of monitoring the effectiveness of a cancer treatment by determining the level of Bsh3 activity or expression in the population of cells following the treatment. A decrease level of Bsh3 activity or expression is indicative of effective treatment. An increase level of Bsh3 activity or expression is indicative of a less effective treatment.

Still another embodiment is a method of enhancing the effectiveness of an antineoplastic agent by treating neoplastic cells with a compound that reduces the level of Bsh3 activity. The compound can be administered before, during, or after the administration of the antineoplastic agent.

### BRIEF DESCRIPTION OF THE DRAWINGS

FIG. 1 is a protein sequence (full-length) alignment of human Bsh3 (top, SEQ ID NO:2) and S. pombe Bsh3p (bottom, SEQ ID NO: 6). The amino acid sequences are compared for maximal alignment. The shaded regions indicate identical amino acid residues among the two proteins.

### DETAILED DESCRIPTION OF THE INVENTION

Embodiments of the invention relate to the discovery that treatment with Bsh3 could rescue a mutation in the yeast homolog of Survivin, bir1p. Since Survivin has a role in mitosis, Bsh3 was expected to have a similar role in mitosis. The following description demonstrates that Bsh3 indeed plays a very important role in mitosis and, thus, is useful for treating and identifying diseases that are related to proliferative growth.

As used herein “Bsh3” includes all homologues, allelic variants, and conservative amino acid substitutions of the protein or nucleic acid encoding the Bsh3 protein. Bsh3 includes the human Bsh3 (“HBsh3” or “Human Bsh3“) and the S. pombe Bsh3. As used herein “Bsh3p” is the protein from S. pombe and “Bsh3⁺” is the gene from S. pombe. In one embodiment, Bsh3 refers to a human Bsh3 protein that has the amino acid sequence shown in SEQ ID NO:2. The term “Bsh3 protein” also includes naturally occurring allelic variants of Bsh3, naturally occurring proteins that have a slightly different amino acid sequence than that specifically recited above. Allelic variants, though possessing a slightly different amino acid sequence than those recited above, will still have the requisite ability to activate cellular mitosis.

As used herein, the Bsh3 family of proteins refers to Bsh3 proteins that have been isolated from organisms in addition to humans. The methods used to identify and isolate other members of the Bsh3 family of proteins are described below.

It was discovered that Bsh3 plays an essential role in chromosome segregation during mitosis. The human homolog of Bsh3⁺, HBsh3, encodes a 185 amino-acid protein that is 30% identical with S. pombe Bsh3p. Human Bsh3-GFP localizes in the nucleus and midbody. Depletion of human Bsh3 in HeLa cells using siRNA (small interfering RNA) resulted in cell growth arrest, chromosome missegregation, micronuclei and irregular nuclei. Similarly, as described below, the yeast Bsh3p protein was found to be involved in mitosis since deletion of yeast Bsh3+ gene resulted in cell elongation, chromosome missegregation, fragmented nuclei, and cut and polyseptation phenotypes.

In the following examples, Bsh3 protein was identified as a suppressor of the birI-46 phenotype and thus was discovered to have an essential function related to the cell cycle. Further, the relationship between Bsh3 and other homologs of bir1p was identified and clarified. It was interesting to note that the homolog of inner centromere protein (INCENP) in S. pombe, pic1⁺ (pombe INCENP), was also found to function as a high-copy suppressor of bir1-46 at 34° C. pic1⁺ and was essential for cell growth. In addition, deletion of pic1⁺ resulted in cell elongation, cut and poly-septation phenotypes. Accordngly, the data herein suggest that Bir1p, Bsh3p and Pic1p function together during chromosome segregation in S. pombe, and that Bsh3p has conserved functions in higher eukaryotes.

As reported below, an ectopically expressed fusion protein of Bsh3p and the green fluorescent protein (Bsh3p-GFP) localized in the nucleus and associated with the elongating mitotic spindle during anaphase. The fusion protein also localized to the central region of mitotic spindle during telophase. The localization of Bsh3p-GFP during cell cycle progression is similar to that of Bir1p, suggesting that Bsh3p and Bir1p regulate a common process during mitosis. Depletion of Bsh3p caused cell elongation, lagging chromosomes and fragmented nuclei, suggesting that Bsh3p is required for proper kinetochore-mitotic spindle attachment. Consistent with this interpretation, the Bsh3+/Bsh3 Δ heterozygous diploid showed a much higher rate of chromosome loss than the Bsh3+/Bsh3 + homozygous diploid. Thus, overexpressed Bsh3p might stabilize the Bir1p ts mutant protein at kinetochore-spindle attachment and thereby rescue the temperature sensitivity of bir1-46.

Conversely, depletion of Bsh3p might destabilize the kinetochore-mitotic spindle attachment during chromosome segregation, and result in chromosome missegregation and other observed phenotypes, such as cell growth arrest, cut and multiple-septation phenotypes. Given that overexpressed Bsh3p-GFP associated with the mitotic spindle, Bsh3p likely interacts with mitotic spindles and stabilizes them. However, overexpression of Bsh3p was not found to rescue the cold-sensitivity of nda2 (β-tubulin) and nda3 (β-tubulin) cold-sensitive mutants.

Bsh3p was found to have homologues in all sequenced eukaryotes, suggesting that Bsh3p has an evolutionarily conserved function. Like Bsh3p in S. pombe, human Bsh3 was found to localize in the nucleus and to the midbody. As discussed below, siRNA-mediated depletion of human Bsh3 in asynchronous HeLa cells arrested cell growth and increased the population of cells with sub-G1 DNA content, presumably due to chromosome missegregation. Consistent with this interpretation, depletion of human Bsh3 resulted in lagging chromosomes in the midbody region during cytokinesis. Thus, depleted cells will have sub-G1 DNA content after cell division. In addition, depletion of human Bsh3 caused micronuclei and irregular nuclei due to chromosome missegregation, which allowed micronuclei to form around lagging chromosome(s). The irregular nuclei could result from nuclear fusion of micronuclei. Unstable kinetochore-mitotic spindle attachments and/or defective cytokinesis, which result from disruption of Survivin, INCENP, CenpA, CenpC and Bub3 function, have been shown to cause micronuclei and irregular nuclei. For these reasons, Bsh3 homologs were found to play a conserved and essential role in stabilizing the kinetochore-mitotic spindle attachment during chromosome segregation.

Accordingly, embodiments of the invention relate to the use of molecules, such as chemicals, proteins, and nucleic acids to modulate the levels of Bsh3 in a cell. Levels of Bsh3 can be modulated by affecting the protein directly, or by altering the expression levels of a nucleic acid encoding Bsh3. Expression includes, but is not limited to, transcription, processing and translation.

In addition, Bsh3 activity can be modulated by directly targeting Bsh3 or by targeting molecules that interact with Bsh3 or which are further downstream of a pathway initiated by Bsh3. Thus, candidate compounds are those that have been found to directly associate with Bsh3, or compounds that associate with proteins or other molecules that bind to Bsh3. Candidate compounds may prevent the expression or activity of Bsh3 or proteins or other molecules that bind to Bsh3 or proteins or other molecules that are further downstream in a regulatory pathway initiated by Bsh3. Compounds may prevent or reduce the binding of Bsh3 to other proteins or molecules.

Another embodiment of the invention relates to methods of identifying candidate compounds that associate with Bsh3 or its binding proteins or proteins downstream. Furthermore, candidate compounds include those that affect the expression of Bsh3, the expression of proteins or other molecules that bind to Bsh3 or the expression of proteins or other molecules that are further downstream in a mitosis regulatory pathway initiated by Bsh3. Any method known to those of skill in the art can be used to identify candidate compounds that affect expression of proteins, including assays that examine transcription, processing or translation. Once candidate compounds are initially identified they can be screened for modulating mitosis using any of the methods described herein.

Bsh3 inhibitors which are identified may be used to reduce the number of undesirable cells in an individual, such as with cancer or tumor cells. Cells are contacted with an effective amount of a Bsh3 inhibitor which leads to reduction of cell growth for the targeted cells. For example, such cells may be present in diseases or conditions which result in the upregulation of Bsh3 and mitosis, including but not limited to cancer and autoimmune diseases.

Activators which are identified for Bsh3 may be used to increase the number of targeted cells in an individual, where the number of these cells is less than desired. Cells are contacted with an effective amount of the activator, which results in increased growth of the target cells. For example, such cells may be present in diseases or conditions which are characterized by a down regulation of Bsh3 and mitosis. In addition, there may be fewer cells than desired under conditions of wound healing, Parkinson's disease or other degenerative diseases, injury, transplant rejection and tissue or organ regeneration.

Candidate compounds which inhibit or activate or modulate Bsh3 can be obtained from a wide variety of sources including libraries of synthetic or natural compounds. For example, numerous means are available for random and directed synthesis of a wide variety of organic compounds and biomolecules, including expression of randomized oligonucleotides and oligopeptides. See, for example, U.S. Pat. No. 5,877,030 to Rebek et al. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, and the like, to produce structural analogs. Candidate compounds can be found among biomolecules including, but not limited to: peptides, saccharides, fatty acids, steroids, purines, pyrimidines, derivatives, structural analogs or combinations thereof.

Embodiments also provide compounds that modulate Bsh3 expression or activity. Such compounds include peptides, peptidomimetics, polypeptides, pharmaceuticals, chemical compounds (inorganic and organic), biological agents, nucleic acids, ribozymes, antibodies and the like. Antibodies and combinatorial compound libraries can also be tested using the methods herein. One class of compounds are organic molecules, Compounds of the invention contain functional groups necessary for structural interaction with proteins, particularly interaction via hydrogen bonds, such compounds typically comprising at least an amine, carbonyl, hydroxyl or carboxyl group, and preferably at least two such functional groups. The compounds also may comprise carbocyclic or heterocyclic structures and/or aromatic or polyaromatic structures substituted with one or more of the above functional groups.

A. Definitions

“Contacting cells” as used herein includes any means for contacting a cell with a compound such that the compound is introduced into the cell. In one example, cells are contacted in such a way that a modulator compound gains access to a population of cells for which mitosis is to be modulated. Contacting is also meant to include introduction of a nucleic acid encoding a modulator compound to a cell, followed by expression in the cell by any means known to those who practice the art. A compound can be introduced into a cell by any known means, including facilitated transport, membrane fusion, liposomes, vesicle mediated transport, and the like.

“Administration” to an individual (e.g. a human) may be by any suitable means, such as by oral, sublingual intravenous, subcutaneous, transcutaneous, intramuscular, intracutaneous, intrathecal, epidural, intraoccular, intracranial, inhalation, rectal, vaginal, and the like. The compound to be administered may be formulated with one or more pharmaceutically acceptable carriers, which can take the form of a cream, lotion, tablet, capsule, pellet, dispersible powder, granule, suppository, syrup, elixir, lozenge, injectable solution, sterile aqueous or non-aqueous solution, suspension or emulsion, patch, and the like. The active compound may be compounded with non-toxic, pharmaceutically acceptable carriers including, glucose, lactose, gum acacia, gelatin, mannitol, starch paste, magnesium trisilicate, talc, corn starch, keratin, colloidal silica, potato starch, urea, dextrans, and the like.

An “effective amount,” refers to a dose sufficient to provide desirable concentrations of the compound taken up, introduced directly into or expressed in cells such that mitosis is affected or modulated either positively or negatively. The increase or decrease in number of cells that would otherwise undergo mitosis in a given cell population is at least about 1%, 5%, 10%, 20%, 40% or more preferably at least about 50% of the cells in that population. The specific effective dose level for any particular subject will depend upon a variety of factors including the disorder being treated, the severity of the disorder, the activity of the specific polypeptide or compositions used, the route of administration, the rate of clearance of the specific polypeptide or composition, the duration of treatment, the drugs used in combination or coincident with the specific polypeptide or composition, the age, body weight, sex, diet and general health of the subject, and like factors well known in the medical arts and sciences. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th ed., Mack Publishing Co., Easton, Pa., 1990. Dosage levels typically fall in the range of about 0.001 up to 100 mg/kg/day; with levels in the range of about 0.05 up to 10 mg/kg/day being preferred.

As used herein, “modulates mitosis” means increasing or decreasing the rate of progression through the cell cycle, or the frequency of cell division over a predetermined time period. Thus, increased mitosis typically leads to an increased number of cells in a population. Conversely, decreased levels of mitosis typically lead to a smaller number of cells in a population, when compared to cell population undergoing mitosis at a standard level. Mitosis can be effected by increasing or decreasing the amount of Bsh3 present in a cell or by increasing or decreasing the activity of the Bsh3 protein. Preferably, the given cell population in which mitosis is to be modulated is found in a tumor or other tissue or group of cells in which a beneficial effect results from the modulation. Also, preferably, the increase or decrease in number of cells that would otherwise undergo mitosis in a given cell population is at least about 1%, 5%, 10%, 20%, 40% or more preferably at least about 50% of the cells in that population.

By “monitor” is meant to assay by any and all applicable methods. Monitoring mitosis is meant to include all means for determining and/or tracking the level of mitosis in a cell.

By “expression” is meant the transcription and translation of a gene. For example the amount and level of Bsh3 mRNA produced from a promoter and/or the amount and level of the Bsh3 protein which is then produced from the mRNA.

By ‘protein activity” is meant the function of a protein, including but not limited to, enzymatic catalysis, transport, signal transduction, control of growth and/or differentiation, binding or association with other molecules, inducing conformational change, localization, kinase activity, phosphatase activity, nuclease activity, transcription and translational modulation.

By “autoimmune reactive cells” is meant cells which are involved in the symptoms and/or pathogenesis of autoimmune diseases. For example, B cells or T cells which recognize self-antigens are autoimmunie reactive cells.

By “associates directly” is meant associates without the requirement of a second protein. For example, a compound might associate directly with Bsh3 by binding to Bsh3 in order to modulate mitosis.

By “associates indirectly” is meant associates with a second protein or other molecule that itself binds to the target molecule. For example, a compound that associates indirectly with Bsh3 might bind to an intermediary to form a complex, and the complex then binds to Bsh3 in order to modulate mitosis in a cell.

By “cancer cells” is meant cells that exhibit a loss of growth control. Cancer cells is meant to include any tumor cells, both malignant and benign.

By “transplantation cells” is meant cells which are replaced, repositioned or added to cells already present in an individual. The cells may be part of a tissue or organ. Examples of such cells include, but are not limited to, stem cells, recombinant cells, cells which are part of an organ, including but not limited to: kidney, liver, lung, heart, parts of the eye, body parts, and veins or arteries, cells which are transformed with a virus, immune cells, and embryonic cells.

By “proliferation potential” is meant the proliferative ability exhibited by a population of cells. Cells with high proliferative potential generally grow and divide rapidly and are less amenable to treatment.

B. Bsh3 Protein

The Bsh3 proteins are preferably in isolated form. As used herein, a protein is said to be isolated when physical, mechanical or chemical methods are employed to remove the Bsh3 protein from cellular constituents that are normally associated with the Bsh3 protein. A skilled artisan can readily employ standard purification methods to obtain an isolated Bsh3 protein.

The Bsh3 proteins further include conservative variants of the Bsh3 proteins herein described. As used herein, a conservative variant refers to alterations in the amino acid sequence that do not adversely affect the ability of the Bsh3 protein to bind to a Bsh3 binding partner and/or to act in cellular mitosis. A substitution, insertion or deletion is said to adversely affect the Bsh3 protein when the altered sequence prevents the Bsh3 protein from associating with a Bsh3 binding partner and/or prevents the Bsh3 protein from acting in cellular mitosis. For example, the overall charge, structure or hydrophobic/hydrophilic properties of Bsh3 can be altered without adversely affecting the activity of Bsh3. Accordingly, the amino acid sequence of Bsh3 can be altered, for example to render the peptide more hydrophobic or hydrophilic, without adversely affecting the activity of Bsh3.

The allelic variants, the conservative substitution variants and the members of the Bsh3 family of proteins, will have the ability to activate or modulate cellular mitosis. Such proteins will ordinarily have an amino acid sequence having at least about 75% amino acid sequence identity with the human Bsh3 sequence, more preferably at least about 80%, even more preferably at least about 90%, and most preferably at least about 95%. Identity or homology with respect to such sequences is defined herein as the percentage of amino acid residues in the candidate sequence that are identical with the known peptides, after aligning the sequences and introducing gaps, if necessary, to achieve the maximum percent homology, and including any conservative substitutions as being homologous. N-terminal, C-terminal or internal extensions, deletions, or insertions into the peptide sequence shall not be construed as affecting homology.

Thus, the Bsh3 proteins include molecules having the amino acid sequences disclosed in SEQ ID NO:2; fragments thereof having a consecutive sequence of at least about 3, 5, 10 or 15 amino acid residues of the Bsh3 protein; amino acid sequence variants of such sequence wherein an amino acid residue has been inserted N- or C-terminal to, or within, the disclosed Bsh3 sequence; amino acid sequence variants of the disclosed Bsh3 sequence, or their fragments as defined above, that have been substituted by another residue.

Contemplated variants further include those containing predetermined mutations by, e.g., homologous recombination, site-directed or PCR mutagenesis, and the corresponding Bsh3 proteins of other animal species, including but not limited to rabbit, rat, murine, porcine, bovine, ovine, equine and non-human primate species, and the alleles or other naturally occurring variants of the Bsh3 family of proteins; and derivatives wherein the Bsh3 protein has been covalently modified by substitution, chemical, enzymatic, or other appropriate means with a moiety other than a naturally occurring amino acid (for example a detectable moiety such as an enzyme or radioisotope). The recombinant Bsh3 protein also can be used to solve the molecular structure of Bsh3 by 2D-NMR, circular dichroism and X-ray crystallography, thus integrating the site-directed mutagenesis approach and the rational design of specific small molecule inhibitors.

As described below, members of the Bsh3 family of proteins can be used: 1) as a target to block Bsh3 mediated activation of cellular mitosis, 2) to identify and isolate binding partners that bind Bsh3, 3) in methods to identify agents that block the association of Bsh3 with a Bsh3 binding partner, 4) as a target to assay for Bsh3 mediated activation of cellular mitosis, 5) as an agent to activate cellular mitosis, administered alone or as part of a combination therapy, 6) as a binding partner in an assay to quantitate circulating levels of anti-Bsh3 antibodies, 7) as an antigen to elicit production of anti-Bsh3 antibodies that in turn can be used in an assay to quantitate circulating levels of Bsh3 and or can be used for immunohistochemical purposes, and 8) as a therapeutic vaccine, or component of a polyvalent vaccine, e.g. an anti-cancer vaccine, 9) to identify antisense and other inhibitors of Bsh3 which could be used as therapeutics for diseases, such as cancer.

C. Anti-Bsh3 Antibodies

Further embodiments provide antibodies that selectively bind to a Bsh3 protein. The anti-Bsh3 antibodies particularly contemplated include monoclonal and polyclonal antibodies as well as fragments containing the antigen binding domain and/or one or more complement determining regions.

Antibodies are generally prepared by immunizing a suitable mammalian host using a Bsh3 protein, or fragment, in isolated or immunoconjugated form (Harlow, Antibodies, Cold Spring Harbor Press, NY (1989)). Methods for preparing immunogenic conjugates of a protein with a carrier such as BSA, KLH, or other carrier proteins are well known in the art. In some circumstances, direct conjugation using, for example, carbodiimide reagents may be used; in other instances linking reagents such as those supplied by Pierce Chemical Co., Rockford, Ill., may be effective.

Administration of the Bsh3 immunogen is conducted generally by injection over a suitable time period and with use of a suitable adjuvant, as is generally understood in the art. During the immunization schedule, titers of antibodies can be taken to determine adequacy of antibody formation.

While the polyclonal antisera produced in this way may be satisfactory for some applications, for pharmaceutical compositions, monoclonal antibody preparations are preferred. Immortalized cell lines which secrete a desired monoclonal antibody may be prepared using the standard method of Kohler and Milstein or modifications which effect immortalization of lymphocytes or spleen cells, as is generally known. The immortalized cell lines secreting the desired antibodies are screened by immunoassay in which the antigen is the Bsh3 peptide. When the appropriate immortalized cell culture secreting the desired antibody is identified, the cells can be cultured either in vitro or by production in ascites fluid.

The desired monoclonal antibodies are then recovered from the culture supernatant or from the ascites supernatant. Fragments of the monoclonals or the-polyclonal antisera which contain the immunologically significant portion can be used as antagonists, as well as the intact antibodies. Use of immunologically reactive fragments, such as the Fab, Fab′, of F(ab′)₂ fragments is often preferable, especially in a therapeutic context, as these fragments are generally less immunogenic than the whole immunoglobulin.

The antibodies or fragments may also be produced, using current technology, by recombinant means. Regions that bind specifically to the desired regions of receptor can also be produced in the context of chimeras or CDR grafted antibodies of multiple species origin.

The antibodies thus produced are useful not only as modulators of the association of Bsh3 with a Bsh3 binding partner, but are also useful in immunoassays for detecting Bsh3 expression/activity and for the purification of Bsh3 and associated binding partners. In some embodiments, the antibodies may be used as therapeutics, e.g., for cancer treatment.

D. Bsh3 Encoding Nucleic Acid Molecules

Further embodiments provide nucleic acid molecules that encode Bsh3, and the related Bsh3 proteins herein described, preferably in isolated form. For convenience, all Bsh3 encoding nucleic acid molecules will be referred to as the Bsh3 encoding nucleic acid molecule, the Bsh3 gene, or Bsh3. As used herein, “nucleic acid” is defined as RNA or DNA that encodes a peptide as defined above, or is complementary to a nucleic acid sequence encoding such peptides, or hybridizes to such a nucleic acid and remains stably bound to it under stringent conditions, or encodes a polypeptide sharing at least 75% sequence identity, preferably at least 80%, and more preferably at least 85%, with the peptide sequences. Specifically contemplated are genomic DNA, cDNA, mRNA and antisense molecules, as well as nucleic acids based on an alternative backbone or including alternative bases whether derived from natural sources or synthesized. Such hybridizing or complementary nucleic acid, however, is defined further as being novel and unobvious over any prior art nucleic acid including that which encodes, hybridizes under appropriate stringency conditions, or is complementary to a nucleic acid encoding a Bsh3 protein.

As used herein, “stringent conditions” are conditions in which hybridization yields a clear and detectable signal. Stringent conditions are those that (1) employ low ionic strength and high temperature for washing, for example, 0.015 M NaCl,0.0015 M sodium titrate, 0.1% SDS at 50° C., or (2) employ during hybridization a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C. Another example is use of 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50:g/ml), 0.1% SDS, and 10% dextran sulfate at 42° C., with washes at 42° C. in 0.2×SSC and 0.1% SDS. A skilled artisan can readily determine and vary the stringency conditions appropriately to obtain a clear and detectable hybridization signal.

As used herein, a nucleic acid molecule is said to be “isolated” when the nucleic acid molecule is substantially separated from contaminant nucleic acid encoding other polypeptides from the source of nucleic acid.

Further embodiments provide fragments of the Bsh3 encoding nucleic acid molecule. As used herein, a fragment of a Bsh3 encoding nucleic acid molecule refers to a small portion of the entire protein encoding sequence. The size of the fragment will be determined by the intended use.

Fragments of the Bsh3 encoding nucleic acid molecules (i.e., synthetic oligonucleotides) that are used as probes or specific primers for the polymerase chain reaction (PCR), or to synthesize gene sequences encoding Bsh3 proteins can easily be synthesized by chemical techniques, for example, the phosphotriester method of Matteucci, et al., J Am Chem Soc (1981) 103:3185-3191 or using automated synthesis methods. In addition, larger DNA segments can readily be prepared by well known methods, such as synthesis of a group of oligonucleotides that define various modular segments of the Bsh3 gene, followed by ligation of oligonucleotides to build the complete modified Bsh3 gene.

The Bsh3 encoding nucleic acid molecules may further be modified so as to contain a detectable label for diagnostic and probe purposes. As described above such probes can be used to identify other members of the Bsh3 family of proteins and as described below, such probes can be used to detect Bsh3 expression and tumor growth potential. A variety of such labels are known in the art and can readily be employed with the Bsh3 encoding molecules herein described. Suitable labels include, but are not limited to, biotin, radiolabeled nucleotides and the like. A skilled artisan can employ any of the art known labels to obtain a labeled Bsh3 encoding nucleic acid molecule.

Specifically, single-stranded diagnostic probes can be used to selectively hybridize to mRNA that encodes Bsh3 protein. Single-stranded probes can be generated using known methods in which one strand of a double-stranded probe is isolated or in which a single stranded RNA probe is generated.

Modifications to the primary structure itself by deletion, addition, or alteration of the amino acids incorporated into the protein sequence during translation can be made without destroying the activity of the protein. Such substitutions or other alterations result in proteins having an amino acid sequence encoded by DNA falling within the contemplated scope of the present invention.

E. Isolation of Other Bsh3 Encoding Nucleic Acid Molecules

As described above, the identification of the human Bsh3 encoding nucleic acid molecule allows a skilled artisan to isolate nucleic acid molecules that encode other members of the Bsh3 family of proteins in addition to the human sequence herein described.

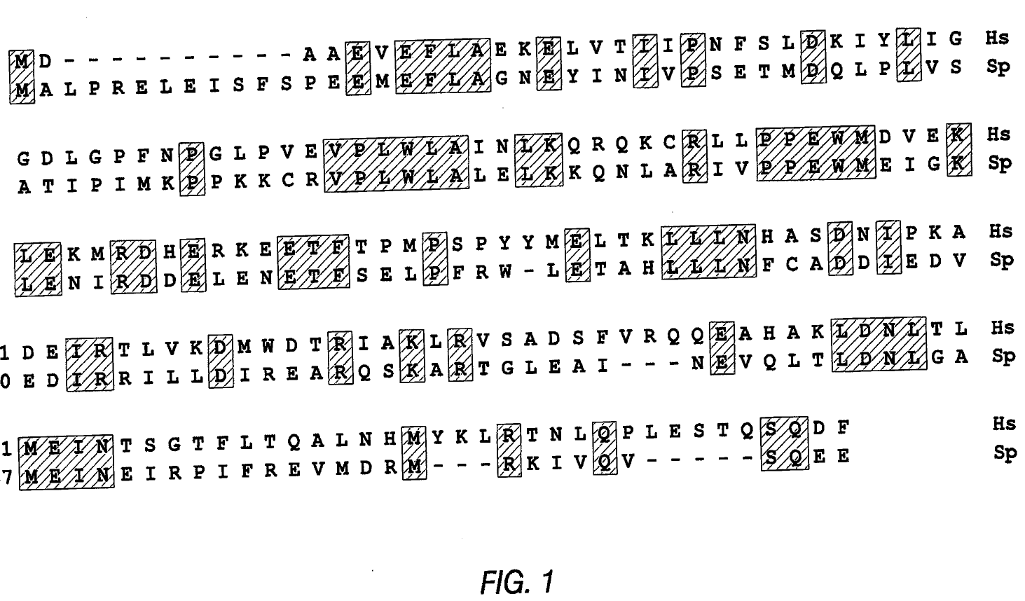

Essentially, a skilled artisan can readily use the amino acid sequence of Bsh3 to generate antibody probes to screen expression libraries prepared from cells. Typically, polyclonal antiserum from mammals such as rabbits immunized with the purified Bsh3 protein (as described below) or monoclonal antibodies can be used to probe a mammalian cDNA or genomic expression library, such as lambda gtll library, to obtain the appropriate coding sequence for Bsh3, or other members of the Bsh3 family of proteins. The cloned cDNA sequence can be expressed as a fusion protein, expressed directly using its own control sequences, or expressed by constructions using control sequences appropriate to the particular host used for expression of the enzyme. FIG. 1 identifies important antigenic and/or putative operative domains found in the Bsh3 protein sequence. Such regions are preferred sources of antigenic portions of the Bsh3 protein for the production of probe, diagnostic, and therapeutic antibodies.

Alternatively, a portion of the Bsh3 encoding sequence herein described can be synthesized and used as a probe to retrieve DNA encoding a member of the Bsh3 family of proteins from any mammalian organisms that contains such a protein. Oligomers containing approximately 18-20 nucleotides (encoding about a 6-7 amino acid stretch) are prepared and used to screen genomic DNA or cDNA libraries to obtain hybridization under stringent conditions or conditions of sufficient stringency to eliminate an undue level of false positives.

Additionally, pairs of oligonucleotide primers can be prepared for use in a polymerase chain reaction (PCR) to selectively clone a Bsh3-encoding nucleic acid molecule. A PCR denature/anneal/extend cycle for using such PCR primers is well known in the art and can readily be adapted for use in isolating other Bsh3 encoding nucleic acid molecules. FIG. 1 identifies regions of the human Bsh3 gene (SEQ ID NO:2) that are particularly well suited for use as a probe or as primers (see also accession number NP_{—}057179).

F. rDNA Molecules Containing a Bsh3 Encoding Nucleic Acid Molecule

Further embodiments provide recombinant DNA molecules (rDNAs) that contain a Bsh3 encoding sequence. As used herein, a rDNA molecule is a DNA molecule that has been subjected to molecular manipulation in vitro. Methods for generating rDNA molecules are well known in the art, for example, see Sambrook et al., Molecular Cloning (1989). In the preferred rDNA molecules, a Bsh3 encoding DNA sequence is operably linked to expression control sequences and/or vector sequences.

The choice of vector and/or expression control sequences to which one of the Bsh3 encoding sequences is operably linked depends directly, as is well known in the art, on the functional properties desired, e.g., protein expression, and the host cell to be transformed. A vector contemplated herein is at least capable of directing the replication or insertion into the host chromosome, and preferably also expression, of the Bsh3 gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a Bsh3 encoding nucleic acid molecule will include a prokaryotic replicon, i.e., a DNA sequence having the ability to direct autonomous replication and maintenance of the recombinant DNA molecule extrachromosomally in a prokatyotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic—or viral promoter capable of directing the expression (transcription and translation) of the Bsh3 encoding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment. Typical of such vector plasmids are pUC8, pUC9, pBR322 and pBR329 available from Biorad Laboratories, (Richmond, Calif.), pPL and pKK223 available from Pharmacia, (Piscataway, N.J.).

Expression vectors compatible with eukaryotic cells, preferably those compatible with vertebrate cells, can also be used to form rDNA molecules that contain a Bsh3 encoding sequence. Eukayotic cell expression vectors are well known in the art and are available from several commercial sources. Typically, such vectors are provided containing convenient restriction sites for insertion of the desired DNA segment. Typical of such vectors are PSVL and pKSV-10 (Pharmacia), pBPV-1/pML2d (International Biotechnologies, Inc.), pTDT1 (ATCC, #31255), the vector pCDM8 described herein, and the like eukaryotic expression vectors.

Eukaryotic cell expression vectors used to construct the rDNA molecules herein may further include a selectable marker that is effective in an eukaryotic cell, preferably a drug resistance selection marker. A preferred drug resistance marker is the gene whose expression results in neomycin resistance, i.e., the neomycin phosphotransferase (neo) gene. Southern et al., J Mol Anal Genet (1982) 1:327-341. Alternatively, the selectable marker can be present on a separate plasmid, and the two vectors are introduced by co-trasfection of the host cell, and selected by culturing in the appropriate drug for the selectable marker.

G. Host Cells Containing an Exogenously Supplied Bsh3 Encoding Nucleic Acid Molecule

A further embodiment provides host cells transformed with a nucleic acid molecule that encodes a Bsh3 protein. The host cell can be either prokaryotic or eukaryotic. Eukaryotic cells useful for expression of a Bsh3 protein are not limited, so long as the cell line is compatible with cell culture methods and compatible with the propagation of the expression vector and expression of the Bsh3 gene product. Preferred eukaryotic host cells include, but are not limited to, yeast, insect and mammalian cells, preferably vertebrate cells such as those from a mouse, rat, monkey or human fibroblastic cell line, the most preferred being cells that do not naturally express a Bsh3 protein. Preferred eukaryotic host cells include the murine IL-3 dependent cell line BaF3, and the like eukaryotic tissue culture cell lines.

Any prokaryotic host can be used to express a Bsh3-encoding rDNA molecule. The preferred prokaryotic host is E. coli.

Transformation of appropriate cell hosts with a rDNA molecule is accomplished by well-known methods that typically depend on the type of vector used and host system employed. With regard to transformation of prokaryotic host cells, electroporation and salt treatment methods are typically employed, see, for example, Cohen et al., Proc Natl Acad Sci USA (1972)69:2110; and Maniatis et al., Molecular Cloning, A Laboratory Manual, Cold Spring Harbor Laboratory, Cold Spring Harbor, N.Y. (1982). With regard to transformation of vertebrate cells with vectors containing rDNAs, electroporation, cationic lipid or salt treatment methods are typically employed, see, for example, Graham et al., Virol (1973) 52:456; Wigler et al., Proc Natl Acad Sci USA (1979) 76:1373-76.

Successfully transformed cells, i.e., cells that contain a rDNA molecule, can be identified by well known techniques. For example, cells resulting from the introduction of an rDNA can be cloned to produce single colonies. Cells from those colonies can be harvested, lysed and their DNA content examined for the presence of the rDNA using a method such as that described by Southern, J Mol Biol (1975) 98:503, or Berent, et al., Biotech (1985) 3:208 or the proteins produced from the cell assayed via an immunological method.

H. Production of Bsh3 or Antisense Bsh3 Using a rDNA Molecule Encoding a Bsh3 or Antisense Bsh3

A further embodiment includes methods for producing a Bsh3 protein that uses one of the Bsh3 encoding nucleic acid molecules herein described. In general terms, the production of a recombinant form of a Bsh3 protein typically involves the following steps.

First, a nucleic acid molecule is obtained that encodes a Bsh3 protein, such as the nucleic acid molecule depicted in FIG. 1. If the Bsh3 encoding sequence is uninterrupted by introns, it is directly suitable for expression in any host. If not, then a spliced form of the Bsh3 encoding nucleic acid molecule can be generated and used or the intron containing nucleic acid molecule can be used in a compatible eukaryotic expression system.

The Bsh3 encoding nucleic acid molecule is then preferably placed in operable linkage with suitable control sequences, as described above, to form an expression unit containing the Bsh3 encoding sequences. The expression unit is used to transform a suitable host and the transformed host is cultured under conditions that allow the production of the Bsh3 protein. Optionally the Bsh3 protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with Bsh3 encoding sequences to produce a Bsh3 protein.

Antisense Bsh3 molecules may be constructed and tested by any method known to one of skill in the art, including but not limited to: designing an antisense molecule using computer designing programs, then testing said antisense oligonucleotides or expressing said antisense oligonucleotides in cells which express Bsh3 or cells with a Bsh3 rDNA molecule. The ability of the antisense to modulate Bsh3 may be identified using any assay known to one of skill in the art. Antisense oligonucleotides may be designed and synthesized by any method known to one of skill in the art.

I. Activation of Mitosis Using Bsh3

As provided above, Bsh3 has been shown to activate cellular mitosis. Accordingly, Bsh3 can be used in methods to activate the growth of cells. In general, cellular mitosis can be increased by contacting a cell with Bsh3.

The are a number of situations in which it is desirable to activate cellular mitosis. For example, growth of tissue culture cells can be activated using the Bsh3 protein. Alternatively, Bsh3 is useful to stimulate the growth of any under-proliferative cells including transplanted cells, tissues or organs.

Hence, Bsh3 protein or Bsh3 gene expression can be used as a means to activate cellular mitosis. The Bsh3 protein can be introduced into a cell in cell culture, for example via liposomal, Penetrin-1 delivery, or inclusion in the cell growth media to activate mitosis. Alternatively, the Bsh3 gene can be introduced and expressed in cells in vivo.

There are pathological conditions characterized by decreased cellular mitosis, for example in accelerated aging disorders. Studies of patients with SMA (Spinal muscular atrophy, a neurodegenerative disease that is thought to be caused by aberrantly decreased mitosis) has demonstrated that the NAIP gene is inactivated and deleted in 75% of these patients. By extension, inactivating mutations in Bsh3 can result in degenerative diseases characterized by aberrantly decreased mitosis and/or under proliferation. In such cases, the Bsh3 gene or the Bsh3 protein can be used to treat the conditions. Accordingly, the Bsh3 protein, or a Bsh3 encoding nucleic acid molecule is administered to an individual as a means of treating reduced mitosis.

J. Methods to Identify Bsh3 Binding Partners

Another embodiment provides methods for use in isolating and identifying binding partners of Bsh3. Specifically, the Bsh3 protein can be used as a capture molecule to identify Bsh3 binding partners. As used herein, a Bsh3 binding partner is a biomolecule (such as a protein, DNA or other cofactor) that binds to Bsh3 and mediates Bsh3 activation of cellular mitosis. The binding partner may upregulate, activate or modulate Bsh3 involvement in cellular mitosis. Alternatively, the binding partner may be an inhibitor of Bsh3.

Any method known to one of skill in the art may be used to identify a Bsh3 binding partner, including but not limited to: one-hybrid and two-hybrid techniques, immunoprecipitation, and chromatography columns (ie: affinity columns of Bsh3 protein). In one embodiment, a Bsh3 protein is mixed with an extract or fraction of a cell that expresses Bsh3 under conditions that allow the association of a binding partner with Bsh3. After mixing, proteins or peptides that have become associated with Bsh3 are separated from the mixture. The binding partner that bound Bsh3 can then be removed and further analyzed.

To identify and isolate a binding partner, the entire Bsh3 protein can be used. Alternatively, a fragment of a Bsh3 protein can be used.

As used herein, a cellular extract refers to a preparation or fraction that is made from a lysed or disrupted cell. The preferred source of cellular extracts will be cells that naturally express Bsh3. Examples of such cells include, but are not limited to tumor cells and embryonic tissues.

A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and the enzyme lysis. In addition, the cellular extract can be prepared from cells that have been freshly isolated from a subject or from cells or cell lines which have been cultured. A skilled artisan can readily adapt methods for preparing cellular extracts in order to obtain extracts for use in the present methods.

Once an extract of a cell is prepared, the extract is mixed with the Bsh3 protein under conditions in which association of Bsh3 with the binding partner can occur. A variety of conditions can be used, the most preferred being conditions that closely resemble conditions found in the cytoplasm of a Bsh3-expressing cell. Features such as osmolarity, pH, temperature, and the concentration of cellular extract used, can be varied to optimize the association of the Bsh3 with the binding partner.

After mixing under appropriate conditions, Bsh3 is separated from the mixture. A variety of techniques can be utilized to separate the mixture. For example, antibodies specific to Bsh3 can be used to immunoprecipitate the Bsh3 and associated binding partner. Alternatively, standard chemical separation techniques such as chromatography and density/sediment centrifugation can be used.

After removal of non-associated cellular constituents found in the extract, the binding partner can be dissociated from the Bsh3 protein using conventional methods. For example, dissociation can be accomplished by altering the salt concentration or pH of the mixture.

To aid in separating associated Bsh3/binding partner pairs from the mixed extract, the Bsh3 protein can be immobilized on a solid support. For example, Bsh3 can be attached to a nitrocellulose matrix or acrylic beads. Attachment of Bsh3 to a solid support further aids in separating peptide/binding partner pair from other constituents found in the extract.

Alternatively, the Bsh3-encoding nucleic acid molecule can be used in a yeast two-hybrid system. The yeast two-hybrid system has been used to identify other protein partner pairs and can readily be adapted to employ the Bsh3 encoding molecules herein described. Alternatively, the Bsh3-encoding nucleic acid molecule can be used in a yeast one-hybrid system or equivalent to identify activators or inhibitors of the transcription and/or translation of Bsh3.

The binding partners described herein may be activators or inhibitors of Bsh3 activity. Thus, depending on the action of the binding partners they may be used as therapeutics for the treatment of cancer, and autoimmune disease (inhibitors) or transplantation rejection, wound healing, burn healing and Parkinson's disease (activators).

The Bsh3 binding partners may then be tested for the ability to modulate mitosis using any of the assays described in section N below.

K. Use of Bsh3 Binding Partners

Once isolated, the Bsh3 binding partners obtained using the above described methods can be used for a variety of purposes. The binding partners can be used as targets to screen for compounds that are activators or inhibitors of these binding partners, to screen for agents that reduce or block the association with Bsh3 or to generate antibodies that bind to the Bsh3 binding partner using techniques known in the art. Antibodies that bind a Bsh3 binding partner can be used to assay Bsh3 activity, as a therapeutic agent to modulate a biological or pathological process mediated by Bsh3, or to purify the binding partner. These uses are described in detail below.

L. Methods to Identify Agents that Block Bsh3 /Binding Partner Interactions

Another embodiment provides methods for identifying agents that reduce or block the association of Bsh3 with a Bsh3 binding partner. Specifically, Bsh3 is mixed with a Bsh3 binding partner in the presence and absence of an agent to be tested. After mixing under conditions that allow association of Bsh3 with the Bsh3 binding partner, the two mixtures are analyzed and compared to determine if the agent reduced or blocked the association of Bsh3 with the Bsh3 binding partner. Agents that block or reduce the association of Bsh3 with the Bsh3 binding partner will be identified as decreasing the amount of association present in the sample containing the tested agent.

As used herein, an agent is said to reduce or block Bsh3/Bsh3 binding partner association when the presence of the agent decreases the extent to which or prevents the Bsh3 binding partner from becoming associated with Bsh3. One class of agents will reduce or block the association by binding to the Bsh3 binding partner while another class of agents will reduce or block the association by binding to Bsh3.

The Bsh3 binding partner used in the above assay can either be an isolated and fully characterized protein or can be a partially characterized protein that binds to Bsh3 or a Bsh3 binding partner that has been identified as being present in a cellular extract. It will be apparent to one of ordinary skill in the art that so long as the Bsh3 binding partner has been characterized by an identifiable property, e.g., molecular weight, the present assay can be used.

Agents that are assayed in the above method can be randomly selected or rationally selected or designed. As used herein, an agent is said to be randomly selected when the agent is chosen randomly without considering the specific sequences involved in the association of the Bsh3 with the Bsh3 binding partner. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. As described above, there are two sites of action for agents that block Bsh3/Bsh3 binding partner interaction: the binding partner contact site on Bsh3 and the Bsh3 contact site on the Bsh3 binding partner. Agents can be rationally selected or rationally designed by utilizing the peptide sequences that make up the contact sites of the Bsh3/Bsh3 binding partner pair. For example, a rationally selected peptide agent can be a peptide whose amino acid sequence is identical to the Bsh3 contact site on the Bsh3 binding partner. Such an agent will reduce or block the association of Bsh3 with the binding partner by binding to the Bsh3 binding partner.

The agents can be, as examples, peptides, small molecules, vitamin derivatives, as well as carbohydrates. A skilled artisan can readily recognize that there is no limit as to the structural nature of the agents. One class of agents are peptide agents whose amino acid sequences are chosen based on the amino acid sequence of the Bsh3 protein.

The peptide agents can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents are antibodies immunoreactive with critical positions of the Bsh3 or Bsh3 binding partner. As described above, antibodies are obtained by immunization of suitable mammalian subjects with peptides, containing as antigenic regions, those portions of the Bsh3 or binding partner, intended to be targeted by the antibodies. Critical regions include the contact sites involved in the association of the Bsh3 with the Bsh3 binding partner.

As discussed below, the important minimal sequence of residues involved in Bsh3 activity define a functional linear domain that can be effectively used as a bait for two-hybrid screening and identification of potential Bsh3-associated molecules. Use of such Bsh3 fragments will significantly increase the specificity of the screening as opposed to using the fall length molecule or the entire BIR domain and is therefore preferred. Similarly, this linear sequence can be also used as an affinity matrix also to isolate Bsh3 binding proteins using a biochemical affinity purification strategy.

M. Uses for Agents that Block the Association of Bsh3 with a Bsh3 Binding Partner

As discussed above, Bsh3 activates cellular mitosis. Agents that reduce or block the interactions of Bsh3 with a Bsh3 binding partner can be used to modulate biological and pathologic processes associated with Bsh3 function and activity such as mitosis.

In detail, a biological or pathological process mediated by Bsh3 can be modulated by administering to a subject an agent that blocks the interaction of Bsh3 with a Bsh3 binding partner.

As used herein, a subject or individual includes any mammal, so long as the mammal is in need of modulation of a pathological or biological process mediated by Bsh3. The term “mammal” is meant an individual belonging to the class Mammalia. The methods and pharmaceuticals are particularly useful in the treatment of human subjects.

As used herein, a biological or pathological process mediated by Bsh3 or Bsh3 binding to a Bsh3 binding partner refers to the wide variety of cellular events mediated by Bsh3. Pathological processes refer to a category of biological processes which produce a deleterious effect. For example, a pathological process mediated by Bsh3 is the uncontrolled activation of cellular mitosis in cells, or overproliferation. This pathological process can be modulated using agents that reduce or block Bsh3/Bsh3 binding partner association or block Bsh3 expression. A further pathological process for which Bsh3 can be used to modulate is reduced cellular mitosis/multiplication. This pathological process can be modulated using Bsh3, a peptidomimetic, an active peptide of Bsh3, or an activator of Bsh3.

As used herein, an agent is said to modulate a pathological process when the agent reduces the degree or severity of the process. For example, an agent is said to modulate tumor cell proliferation when the agent decreases the rate or extent of cell division.

N. Methods to Identify Inhibitors and/or Activators of Bsh3 Activity or Expression

As used herein, modulation of mitosis means increasing or decreasing the rate of progression through the cell cycle, or the frequency of cell division over a predetermined time period. This can be effected by increasing or decreasing the amount of Bsh3 present in a cell or by increasing or decreasing the activity of the Bsh3 protein. Preferably, the given cell population in which mitosis is to be modulated is found in a tumor or other tissue or group of cells in which a beneficial effect results from the modulation. Also, preferably, the increase or decrease in number of cells that would otherwise undergo mitosis in a given cell population is at least about 1%, 5%, 10%, 20%, 40% or more preferably at least about 50% of the cells in that population. Inhibitors or activators may be inhibitors or activators of transcription or translation of Bsh3. Alternatively, they may be inhibitors or activators of the activity of the Bsh3 protein. Such inhibitors and/or activators may be identified as set out in section J above.

Assays to identify inhibitors or activators of Bsh3 activity or expression involve contacting cells or introducing into cells agents and thereafter monitoring mitosis. Numerous assays to measure mitosis or cell proliferation are known to those who practice the art, including but not limited to: Tritiated Thymidine assays, BrdU (such as APO-BRDU, Phoenix Flow Systems), and MTS assays (a tetrazolium salt), alone or in combination with FACS analysis. Alternatively, the transcription of Bsh3 may be monitored using any methods known to one of skill in the art, including but not limited to: Northern blot technology, quantitative PCR methods, and any kits available from vendors. The translation of Bsh3 may be monitored using methods such as Western blot related techniques, immunoprecipitation, and Bsh3 activity assays (such as mitosis or proliferation assays).

In addition to the foregoing discussion, specific examples of mitosis assays are also provided in the following references. Assays for mitosis in lymphocytes are disclosed by: Li et al., “Induction of mitosis in uninfected lymphocytes by HIV-1 Tat protein”, Science 268:429-431, 1995; Gibellini et al., “Tat-expressing Jurkat cells show an increased resistance to different apoptotic stimuli, including acute human immunodeficiency virus-type 1 (HIV-1) infection”, Br. J. Haematol. 89:24-33, 1995; Martin et al., “HIV-1 infection of human CD.sup.+ T cells in vitro. Differential induction of mitosis in these cells.” J. Immunol. 152:330-42, 1994; Terai et al., “Mitosis as a mechanism of cell death in cultured T lymphoblasts acutely infected with HIV-1”, J. Clin Invest. 87:1710-5, 1991; Dhein et al., “Autocrine T-cell suicide mediated by APO-1/(Fas/CD95)11, Nature 373:438-441, 1995; Katsikis et al., “Fas antigen stimulation induces marked mitosis of T lymphocytes in human immunodeficiency virus-infected individuals”, J. Exp. Med. 1815:2029-2036, 1995; Westendorp et al., Sensitization of T cells to CD95-mediated mitosis by HIV-1 Tat and gp120”, Nature 375:497, 1995; DeRossi et al., Virology 198:234-44, 1994.

Assays for mitosis in fibroblasts are disclosed by: Vossbeck et al., “Direct transforming activity of TGF-beta on rat fibroblasts”, Int. J. Cancer 61:92-97, 1995; Goruppi et al., “Dissection of c-myc domains involved in S phase induction of NIH3T3 fibroblasts”, Oncogene 9:1537-44, 1994; Fernandez et al., “Differential sensitivity of normal and Ha-ras transformed C3H mouse embryo fibroblasts tumor necrosis factor; induction of bcl-2, c-myc, and manganese superoxide dismutase in resistant cells”, Oncogene 9:2009-17, 1994; Harrington et al., “c Myc-induced mitosis in fibroblasts is inhibited by specific cytokines”, EMBO J., 13:3286-3295, 1994; Itoh et al., “A novel protein domain required for mitosis. Mutational analysis of human Fas antigen”, J. Biol. Chem. 268:10932-7, 1993.

Assays for mitosis in neuronal cells are disclosed by: Melino et al., “Tissue transglutaminase and mitosis: sense and antisense transfection studies with human neuroblastoma cells”, Mol. Cell Biol. 14:6584-6596, 1994; Rosenblaum et al., “Evidence for hypoxia-induced, programmed cell death of cultured neurons”, Ann. Neurol. 36:864-870, 1994; Sato et al., “Neuronal differentiation of PC12 cells as a result of prevention of cell death by bcl-2”, J. Neurobiol. 25:1227-1234, 1994; Ferrari et al., “N-acetylcysteine D- and L-stereoisomers prevents apoptotic death of neuronal cells”, J. Neurosci. 1516:2857-2866, 1995; Talley et al., “Tumor necrosis factor alpha-induced mitosis in human neuronal cells: protection by the antioxidant N-acetylcysteine and the genes bcl-2 and crma”, Mol. Cell Biol. 1585:2359-2366, 1995; Talley et al., “Tumor Necrosis Factor Alpha-Induced Mitosis in Human Neuronal Cells: Protection by the Antioxidant N-Acetylcysteine and the Genes bcl-2 and crma “Mol. Cell. Biol. 15:2359-2366, 1995; Walkinshaw et al., “Induction of mitosis in catecholaminergic PC12 cells by L-DOPA. Implications for the treatment of Parkinson's disease,” J. Clin. Invest. 95:2458-2464, 1995.

Assays for mitosis in insect cells are disclosed by: Clem et al., “Prevention of mitosis by a baculovirus gene during infection on insect cells”, Science 254:1388-90, 1991; Crook et al., “An mitosis-inhibiting baculovirus gene with a zinc finger-like motif”, J. Virol: 67:2168-74, 1993; Rabizadeh et al., “Expression of the baculovirus p35 gene inhibits mammalian neural cell death”, J. Neurochem. 61:2318-21, 1993; Birnbaum et al., “An mitosis inhibiting gene from a nuclear polyhedrosis virus encoding a polypeptide with Cys/His sequence motifs”, J. Virol. 68:2521-8, 1994; Clem et al., Mol. Cell. Biol. 14:5212-5222, 1994.

O. Administration of Agents that Affect Bsh3 Activity

Cancer cells are characterized by more rapid cell division and proliferation than observed in most healthy cells, and many anti cancer agents operate by inhibiting cell division. Since cancer cells divide more rapidly than do healthy cells, cancer cells are preferentially killed by anti cancer agents which inhibit mitosis. Such compounds are called “antimitotic”. There continues to be a need for new anti cancer drugs.

Embodiments of the invention include any method that utilizes a substance (anti-Bsh3 compound) which inhibits or acts on Bsh3 to inhibit mitosis, including: antisense Bsh3, Bsh3 antibodies, and Bsh3 inhibitors to control the growth of cancer cells. The anti-Bsh3 compounds may be used to control cancer in mammals when taken up in a pharmaceutically acceptable carrier at a pharmacologically effective concentration. Anti-Bsh3 compounds include antisense, inhibitors of the interaction of Bsh3 with its binding partners, inhibitors of expression of Bsh3, peptide inhibitors, chemical and small molecule inhibitors, ribozymes and any other types of inhibitors known to one of skill in the art. As used herein, the term “mammal” includes so called warm blooded animals such as dogs, rats, mice, cats, guinea pigs, horses, cattle, sheep, and primates including humans. As used herein, the term “controlling the growth” means slowing, arresting, interrupting, or stopping the growth and metastases of rapidly growing tissue, such as a tumor, in a mammal, it being understood that treatment does not generally provide a “cure” in the sense that the tissue is necessarily destroyed or totally eliminated.

Also within the scope is a method for preventing cell reproduction by directly treating cells with one or more of the anti-Bsh3 compounds described herein. More generally, the treatment of tumors and other diseases responsive to the inhibition of cell mitosis is within the scope. As used herein, the term “tumor” means both benign and malignant tumors or neoplasms and includes melanomas, lymphomas, leukemias and sarcomas. As used herein, the term “tumor” is to be construed as encompassing only those specific tumor tissues which are sensitive to treatment with compounds described herein.

Pharmaceutically acceptable acid addition salts of compounds described herein are also useful in treating disease. The term “pharmaceutically acceptable acid addition salts” is intended to include any non toxic organic or inorganic acid addition salts of basic forms of the compounds described herein. In general, compounds having basic groups may form acid addition salts. When several basic groups are present, mono or poly salts may be formed. For example compounds such as those containing a pyridine ring or an amino substituent, may be reacted with a pharmaceutically acceptable acid, and the resulting acid addition salt may be administered. Suitable inorganic acids for use in preparing acid addition salts are well known to the art of pharmaceutical formulation and include hydrochloric, hydrobromic, hydroiodic, sulfuric, nitric, and phosphoric acids, and acid metal salts such as sodium monohydrogen orthophosphate and potassium hydrogen sulfate. Examples of organic acids which form suitable salts include mono, di, and tricarboxylic acids, such as acetic, glycolic, lactic, pyruvic, malonic, fumaric, benzoic, citric, maleic, tartaric, succinic, gluconic, ascorbic, sulfamic, oxalic, pamoic, hydroxymaleic, hydroxybenzoic, phenylacetic, salicylic, methanesulfonic, ethanesulfonic, 2 hydroxyethanesulfonic, benzenesulfonic, or 2 phenoxybenzoic acids or mixtures thereof. (See, for example Berge, et al., “Pharmaceutical Salts,” in J. Pharm. Sci., 66:1 19 (1977)). Acid addition salts may be prepared by standard techniques such as by dissolving the free base in aqueous or aqueous alcohol solution or other suitable solvent containing the appropriate acid and isolating by evaporating the solution or by reacting the free base in an organic solvent in which case the salt separates directly or can be obtained by concentration of the solution. In general, acid addition salts are crystalline materials which are more soluble in water than the free base. As a specific example, the hydrochloride salt of compound 44 (described in Table 3, below) may be prepared by dissolving the compound in anhydrous ethyl ether, bubbling in dry hydrogen chloride gas, filtering, and drying the resultant precipitate.

For pharmaceutical use, the compounds described herein may be taken up in pharmaceutically acceptable carriers, such as, for example, solutions, suspensions, tablets, capsules, ointments, elixirs and injectable compositions. Pharmaceutical preparations may contain from 0.1% to 99% by weight of active ingredient. Preparations which are in single dose form, “unit dosage form”, preferably contain from 20% to 90% active ingredient, and preparations which are not in single dose form preferably contain from 5% to 20% active ingredient. As used herein, the term “active ingredient” refers to compounds described herein, salts thereof, and mixtures of compounds described herein with other pharmaceutically active compounds. Dosage unit forms such as, for example, tablets or capsules, typically contain from about 0.05 to about 1.0 g of active ingredient. Pharmaceutical preparations may be administered orally, parenterally, or topically.

Pharmaceutical preparations containing compounds described herein may be prepared by methods known to those skilled in the art, such as, for example, conventional mixing, granulating, dissolving, or lyophilizing. Oral dosage forms include capsules, pills, tablets, troches, lozenges, melts, powders, solutions, suspensions and emulsions. For oral dosage forms, for example, the compounds may be combined with one or more solid pharmaceutically acceptable carriers, optionally granulating the resulting mixture. Pharmaceutically acceptable adjuvants may optionally be included, such as, for example, flow regulating agents and lubricants. Suitable carriers include, for example, fillers such as sugars, cellulose preparations, calcium phosphates; and binders such as methylcellulose, hydroxymethylcellulose, and starches, such as, for example, maize starch, potato starch, rice starch, and wheat starch. Examples of orally administrable pharmaceutical preparations are dry filled capsules consisting of gelatin, and soft sealed capsules consisting of gelatin and a plasticizer such as glycerol or sorbitol. The dry filled capsules may contain the active ingredient in the form of a granulate, for example in admixture with fillers, binders, glidants, and stabilizers. In soft capsules, the active ingredient is preferably dissolved or suspended in a suitable liquid adjuvant, such as, for example, a fatty oil, paraffin oil, or liquid polyethylene glycol, optionally in the presence of stabilizers. Other oral adminstrable forms include syrups containing active ingredient, for example, in suspended form at a concentration of from about 1% to 20%, preferably about 10%, or in a similar concentration that provides a suitable single dose when administered, for example, in measures of 5 to 10 milliliters. Suitable excipients for use in oral liquid dosage forms include diluents such as water and alcohols, for example ethanol, benzyl alcohol and polyethylene alcohols, either with or without the addition of a pharmaceutically acceptable surfactant, suspending agent, or emulsifying agent. Also suitable are powdered or liquid concentrates for combining with liquids such as milk. Such concentrates may also be packed in single dose quantities.

Suitable rectally administrable pharmaceutical preparations include, for example, suppositories consisting of a combination of active ingredient with a suppository base material. Suitable suppository base materials include, for example, natural or synthetic triglycerides, paraffin hydrocarbons, polyethylene glycols, and alkanols.

The compounds described herein may be administered parenterally, that is, subcutaneously, intravenously, intramuscularly, or interperitoneally, as injectable dosages of the compound in a physiologically acceptable diluent with a pharmaceutical carrier. Solutions for parenteral administration may be in the form of infusion solutions. A pharmaceutical carrier may be, for example, a sterile liquid or mixture of liquids such as water, saline, aqueous dextrose and related sugar solutions, an alcohol such as ethanol, glycols such as propylene glycol or polyethylene glycol, glycerol ketals such as 2,2 dimethyl 1,3 dioxolane 4 methanol, ethers such as poly(ethyleneglycol)400, oils, fatty acids, fatty acid esters or glycerides, with or without the addition of a pharmaceutically acceptable surfactant such as a soap or detergent, suspending agent such as pectin, carbomers, methylcellulose, hydroxypropylmethylcellulose, or carboxymethylcellulose, or emulsifying agent or other pharmaceutically acceptable adjuvants. Examples of oils which may be used in parenteral formulations include petroleum, animal, vegetable, or synthetic oils such as, for example, peanut oil, soybean oil, sesame oil, cottonseed oil, corn oil, olive oil, petrolatum, and mineral oil. Suitable fatty acids include, for example, oleic acid, stearic acid, and isostearic acid. Suitable fatty acid esters include ethyl oleate and isopropyl myristate. Suitable soaps include alkaline metal, ammonium and triethanolamine salts of fatty acids. Suitable detergents include cationic detergents such as dimethyl dialkyl ammonium halides and alkyl pyridinium halides; anionic detergents such as alkyl, aryl and olefin sulfonates, monoglyceride sulfates and sulfosuccinates; nonionic detergents such as fatty amine oxides, fatty acid alkanolamides and polyoxyethylenepropylene copolymers; and amphoteric detergents such as alkyl alpha. aminopropionates and 2 alkylimidazoline quaternary ammonium salts; as well as mixtures of detergents. Parenteral preparations will typically contain from about 0.5% to about 25% by weight of active ingredient in solution. Preservatives and buffers may also be used advantageously. Injection suspensions may include viscosity increasing substances such as, for example, sodium carboxymethylcellulose, sorbitol or dextran, and may also include stabilizers. In order to minimize irritation at the site of injection, injectable compositions may contain a non ionic surfactant having a hydrophile lipophile balance (HLB) of from about 12 to about 17. The quantity of surfactant in such formulations ranges from about 5% to about 15% by weight. The surfactant may be a single component having the above HLB or a mixture of two or more components having the desired HLB. Particular examples of useful surfactants include polyethylene sorbitan fatty acid esters, such as, for example, sorbitan monooleate.

It is generally known that therapeutic agents used in the treatment of disease such as cancer may be used in conjunction with other therapeutic agents or therapies known to be useful in treatment of the disease. In particular, the anti-Bsh3 and anti-mitotic compounds described herein may be used in such conjunctive therapy. For example, the administration of a compound described herein may be used in conjunction with the excision of a tumor or with irradiation therapy, immunotherapy, or local heat therapy. Compounds described herein may advantageously be administered in conjunction with a chemical cytotoxic agent known to be useful for the treatment of tumors. It is known to those skilled in the art that combination therapy may provide enhanced therapeutic effects including slowing or prevention of regrowth of a tumor. It is also known to those skilled in the art that when using combination therapy, it is essential to avoid undesirable interactions between compounds or adverse effects on a patient due to inappropriate combinations of compounds. Combination therapy may also allow for smaller doses or fewer individual doses of cytotoxic agents to be used. Such combination therapy utilizing the compounds described herein is contemplated in the method.

It will be understood that the amount of compound actually administered will be determined by a physician or veterinarian in light of the relevant circumstances, including the condition to be treated and the chosen route of administration. At the discretion of a physician or veterinarian, the compounds may be administered therapeutically or prophylactically. Factors included in determining the dosage level include the nature and severity of the disease, the disease stage, and, when administered systemically, the age, sex, size and weight of the subject. The total amount of active ingredient administered will generally range from about 1 milligram (mg) per kilogram (kg) of subject weight to about 100 mg/kg, and preferably from about 3 mg/kg to about 25 mg/kg. A unit dosage may contain from about 25 mg to 1 gram of active ingredient, and may be administered one or more times per day.

The compounds may be applied topically to treat skin cancers. Skin cancers include, for example, cutaneous T cell lymphoma, Sezany lymphoma, xeroderma pigmentosium, ataxia telangiectasia and Bloom's syndrome. A sufficient amount of a preparation containing a compound herein is applied to cover a lesion or affected area. An effective concentration of active agent for topical application is generally within the range of from 10³ moles/liter (M) to 10⁵ M, preferably 10⁴ M. The compounds may be taken up in a suitable carrier for topical application such as, for example, ointments, solutions and suspensions.

It will be understood by those skilled in the art that the compounds herein may be useful in treating diseases, other than cancer, which may be inhibited by antimitotic agents. Treatment of such diseases may involve the use of a combination of pharmaceutical agents and the compounds used in the method may be useful in such combination therapies. For example, treatment of gout typically involves the use of anti-inflammatory drugs in combination with anti-mitotic agents or anti-neoplastic agents such as colchicine, vinblastine and vincristine. The compounds are also expected to be useful in the treatment of gout and may be used in conjunction with anti-inflammatory drugs.

The agents, whether they be agents that block Bsh3/binding partner association or the Bsh3 protein, can be administered via parenteral, subcutaneous, intravenous, intramuscular, intraperitoneal, transdermal, or buccal routes. Alternatively, or concurrently, administration may be by the oral route. Preferably, the agents are delivered directly to the population of cells in which mitosis is to be modulated by means such as injection or perfusion and the agent is taken up by the cells. If the agent is administered systemically, it can be targeted to a particular population of cells by use of methods known to those who practice the art, such as, by constructing chimeric molecules with antibodies or antibody fragments specific for target cells, attaching to receptors or ligands displayed by the target population of cells or encapsulated in liposomes coated with appropriate antibodies, receptors or ligands. Nucleic acids encoding agents can be delivered to target cell populations by target specific vectors, such as viruses. The dosage administered will be dependent upon the age, health, and weight of the recipient, kind of concurrent treatment, if any, frequency of treatment, and the nature of the effect desired. For example, to treat tumor cells as a means of blocking Bsh3 activation of mitosis, an agent that blocks Bsh3 expression or the interaction of Bsh3 with a binding partner, is administered systemically or locally to the individual being treated. As described below, there are many methods that can readily be adapted to administer such agents.

A further embodiment is compositions containing Bsh3 or one or more agents that block Bsh3/binding partner association. While individual needs vary, a determination of optimal ranges of effective amounts of each component is within the skill of the art. Typical dosages comprise 0.1 to 100 μg/kg body wt. The preferred dosages comprise 0.1 to 10 μg/kg body wt. The most preferred dosages comprise 0.1 to 1 μg/kg body wt.

In addition to the pharmacologically active agent, the compositions may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration may be formulated for enteral, parenteral or topical administration. Indeed, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

In practicing the methods, the compounds may be used alone or in combination, or in combination with other therapeutic or diagnostic agents. In certain preferred embodiments, the compounds may be co-administered along with other compounds typically prescribed for these conditions according to generally accepted medical practice, such as chemotherapeutic agents.

P. Combination Therapy

Bsh3, as well as agents that modulate Bsh3 activity, can be provided alone, or in combination with other agents that modulate a particular biological or pathological process. For example, agents that reduce Bsh3 mediated mitosis can be administered in combination with other anti-cancer agents in methods to control cancer cell growth. In one embodiment of the invention, compounds that inhibit Bsh3 activity are used to increase the effectiveness of anti-cancer (antineoplastic) compounds.

Alternatively, Bsh3 can be administered with other protective agents as a means for increasing cellular mitosis. As used herein, two agents are said to be administered in combination when the two agents are administered simultaneously or are administered independently in a fashion such that the agents will act at the same time.

Inhibition of Bsh3 activity/expression can be used in combination with conventional chemotherapies. The timing for using a chemotherapeutic agent in combination with inhibiting Bsh3 activity/expression depends upon chemotherapeutic agent used and the tumor cell type treated. Examples of chemotherapeutic agents that can be used in combination with agents the effect Bsh3 activity/expression, includes, but is not limited to alkylating agents, such as cyclophosphamide (CTX; cytoxan), chlorambucil (CHL; leukeran), cisplatin (CisP; platinol) busulfan (myleran), melphalan, carmustine (BCNU), streptozotocin, triethylenemelamine (TEM), mitomycin C, and the like alkylating agents; anti-metabolites, such as methotrexate (MTX), etoposide (VP16; vepesid) 6-mercaptopuine (6MP), 6-thiocguanie (6TG), cytarabine (Ara-C), 5-fluorouracil (5FU), dacarbazine (DTIC), and the like anti-metabolites; antibiotics, such as actinomycin D, doxorubicin (DXR; adriamycin), daunorubicin (daunomycin), bleomycin, mitbramycin and the like antibiotics; alkaloids, such as vinca alkaloids such as vincristne (VCR), vinblastine, and the like; and other antitumor agents, such as taxol and taxol derivatives, the cytostatic agents glucocorticoids such as dexamethasone (DEX; decadron) and corticosteroids such as prednisone, nucleoside enzyme inhibitors such as hydroxyurea, amino acid depleting enzymes such as asparaginase, and the like diverse antitumor agents.

The use of the cytotoxic agents described above in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using the present histoculture methods. Generally, the dosage will be reduced compared to the amount used in the absence of agents the effect Bsh3 activity/expression.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

Q. Methods for Identifying Bsh3 Expression and Bsh3-Mediated Activation of Mitosis

A further embodiment of the invention provides methods for identifying cells involved in Bsh3-mediated activation of mitosis as well as techniques that can be applied to diagnose biological and pathological processes associated with Bsh3 activity, the progression of such conditions, the susceptibility of such conditions to treatment and the effectiveness of treatment for such conditions. Specifically, Bsh3-mediated activation of mitosis can be identified by determining whether the Bsh3 protein is expressed in a cell. Cells over-expressing Bsh3 are considered to be have increased or overexpressed cellular mitosis.

A variety of immunological and nucleic acid techniques can be used to determine if the Bsh3 protein, or a Bsh3 encoding mRNA, is produced in a particular cell. In one example, an extract of cells is prepared. The extract is then assayed to determine whether Bsh3 is expressed in the cell. The degree of expression provides a measurement of the amount of mitosis (or proliferation). An increase in expression is a measurement of increased mitosis.

The measurement of Bsh3 expression can be used as a marker for a variety of purposes. In tumors, the presence of Bsh3 expression correlates with the proliferative potential of the tumor. In the Examples, it is shown that lymphomas display varying levels of Bsh3 expression; lymphomas showing little to no Bsh3 expression are low grade lymphomas that can be effectively treated while lymphomas showing high levels of Bsh3 expression are high grade aggressive lymphomas that typically cannot be effectively treated. Accordingly, the level of Bsh3 expression in a lymphoma, or other tumor, can be used as a predictive measurement of the aggressiveness and treatability of the tumor: the higher the level of Bsh3 expression, the higher the aggressiveness of the tumor and the more difficult the treatment will be.

For example, to determine a tumor's proliferative potential or easy/prognosis of treatment, an extract is made of the tumor cells and the extract is then analyzed, for example, by gel electrophoresis, to determine whether a Bsh3 protein is present. The presence and level of Bsh3 correlates with the proliferative potential of the cancer and the ease of treatment. Alternatively, as described above, single-strand probes can be used to identify Bsh3-encoding mRNA in the cellular extracts.

In addition to being a marker of tumor aggressiveness and treatment potential, Bsh3 expression can be used as a measurement of the effectiveness of anti-tumor therapy. In the Examples, it is shown that HL-60, a promylocytic cell line, had high levels of Bsh3 expression. Treatment of HL60 cells with retenoic acid, and anti-cancer agent that acts by causing the differentiation of tumor cells, resulted in a reduction and elimination of Bsh3 expression. The reduction in expression correlated with the degree of differentiation, the greater the differentiation, the lower the level of Bsh3 expression. Accordingly, Bsh3 expression can be used to measure the effectiveness of anti-tumor treatment: if Bsh3 expression decreases during treatment, the treatment protocol is effective and can be continued, whereas if Bsh3 expression remains unaltered, a different therapeutic regime or protocol needs to be performed.

R. Animal Models

The Bsh3 gene and the Bsh3 protein can serve as a target for gene therapy in a variety of contexts. For example, in one application, Bsh3-deficient non-human animals can be generated using standard knock-out procedures to inactivate a Bsh3 gene or, if such animals are non-viable, inducible Bsh3 antisense molecules can be used to regulate Bsh3 activity/expression. Alternatively, an animal can be altered so as to contain a Bsh3 or antisense-Bsh3 expression unit that directs the expression of Bsh3 or the antisense molecule in a tissue specific fashion In such a uses, a non-human mammal, for example a mouse or a rat, is generated in which the expression of the Bsh3 gene is altered by inactivated or activation. This can be accomplished using a variety of art-known procedures such as targeted recombination. Once generated, the Bsh3-deficient animal, the animal that expresses Bsh3 in a tissue specific manner, or an animal that expresses an antisense molecule can be used to 1) identify biological and pathological processes mediated by Bsh3, 2) identify proteins and other genes that interact with Bsh3, 3) identify agents that can be exogenously supplied to overcome Bsh3 deficiency and 4) serve as an appropriate screen for identifying mutations within Bsh3 that increase or decrease activity.

For example, it is possible to generate transgenic mice expressing the human minigene for Bsh3 in a tissue specific-fashion and test the effect of over-expression of the protein in cells that normally do not contain Bsh3. This strategy has been successfully used for another family of mitosis inhibitors, namely bcl-2 (Veis et al., Cell (1993) 75:229). Such an approach can readily be applied to the Bsh3 protein and can be used to address the issue of a potential beneficial effect of Bsh3 in a specific tissue area, for example in the case of transplanted cells.

S. Bsh3 Gene Therapy

In another embodiment, genetic therapy can be used as a means for modulating a Bsh3-mediated biological or pathological process or for cancer treatment. For example, in tumor therapy, it may be desirable to introduce into the subject being treated a genetic expression unit that encodes an inhibitor of Bsh3 expression or the Bsh3 polypeptide or an active variant. The expression product can either be constitutively produced or inducible within a cell or specific target cell. This allows a continual or inducible supply of a modulator of Bsh3 expression within the subject. Expressing Bsh3 or increasing Bsh3 expression allows for the activation of growth for example in a transplant. Similarly, cells may be genetically engineered to express Bsh3. In particular, Bsh3 or a Bsh3 activator may be used to sustain neuronal cells that undergo mitosis in the course of a neurodegenerative disease, lymphocytes (i.e., T cells and B cells), or cells that have been injured by ischemia. Alternatively, it may be used for transplantation of cells and/or organs.

The level of Bsh3 gene expression may correlate with the level of mitosis and/or proliferation. Thus, anti-Bsh3 genes also find use in anti-proliferative gene therapy. Retroviral vectors, adenoviral vectors, adeno-associated viral vectors, or other viral vectors with the appropriate tropism for cells undergoing overproliferation may be used as a gene transfer delivery system for a therapeutic Bsh3 gene construct. Numerous vectors useful for this purpose are generally known (Miller, Human Gene Therapy 15-14, 1990; Friedman, Science 244:1275-1281, 1989; Eglitis and Anderson, BioTechniques 6:608-614, 1988; Tolstoshev and Anderson, current opinion in biotechnology 1:55-61, 1990; Sharp, The Lancet 337:1277-1278, 1991; Cornetta et al., i Nucleic Acid Research and Molecular Biology 36:311-322, 1987; Anderson, Science 226:401-409, 1984; Moen, blood Cells 17:407-416, 1991; Miller et al., Biotechniques 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches may also be employed for the introduction of therapeutic DNA into cells otherwise predicted to undergo mitosis. For example, Bsh3 may be introduced into a neuron or a T cell by lipofection (Feigner et al., Proc. Natl. Acad. Sci. USA 84:7413, 1987; Ono et al., Neurosci. Lett. 117:259, 190; Brigham et al., Meth. Enz. 101:512, 1983), asialorosonucoid-polylysine conjugation (Wu et al., J. Biol. Chem. 263:14621, 1988; Wu et al., J. Biol. Chem. 264:16985, 1989); or, less preferably, microinjection under surgical conditions (Wolff et al., Science 247:1465, 1990).

For any of the methods of application described above, the therapeutic Bsh3 nucleic acid construct is preferably applied to the site of the predicted mitosis event (for example, by injection). However, it may also be applied to tissue in the vicinity of the predicted mitosis event or to a blood vessel supplying the cells predicted to undergo mitosis.

In the constructs described, Bsh3 cDNA expression can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in neural cells, T cells, or B cells may be used to direct Bsh3 expression. The enhancers used could include, without limitation, those that are characterized as tissue- or cell-specific in their expression. Alternatively, if a Bsh3 genomic clone is used as a therapeutic construct (for example, following its isolation by hybridization with the Bsh3 cDNA described above), regulation may be mediated by the cognate regulatory sequences or, if desired, by regulatory sequences derived from a heterologous source, including any of the promoters or regulatory elements described above.

T. Use of the Bsh3 Promoter to Direct Gene Expression

A further embodiment provides the promoter of the Bsh3 gene in a form that can be used in generating expression vectors. Specifically, the Bsh3 promoter, identified as being 5′ from the ATG start codon in of Bsh3, can be used to direct the expression of an operably linked protein encoding DNA sequence. Since the Bsh3 promoter does not have a TATA box, a skilled artisan would use a 5′ fragment. As discussed below, transfection of 3T3 cells with the c-myc oncogene results in the up-regulation of Bsh3 mRNA as detected by Northern blots. Accordingly, DNA encoding anti-tumor polypeptides under the control of the Bsh3 promoter could be used to transfect tumor cell where they would be expressed. A skilled artisan can readily use the Bsh3 promoter in expression vectors using methods known in the art.

U. Preventative Anti-mitotic Therapy

In a patient diagnosed to be heterozygous for a Bsh3 mutation or to be susceptible to Bsh3 mutations (even if those mutations do not yet result in alteration or loss of Bsh3 biological activity), or a patient diagnosed with a degenerative disease (e.g., motor neuron degenerative diseases such as SMA or ALS diseases), or diagnosed as HIV positive, any of the disclosed therapies may be administered before the occurrence of the disease phenotype: For example, the therapies may be provided to a patient who is HIV positive but does not yet show a diminished T cell count or other overt signs of AIDS. In particular, compounds shown to increase Bsh3 expression or Bsh3 biological activity may be administered by any standard dosage and route of administration. Alternatively, gene therapy using a Bsh3 expression construct may be undertaken to reverse or prevent the cell defect prior to the development of the degenerative disease.

The methods may be used to reduce or diagnose the disorders described herein in any mammal, for example, humans, domestics pets, or livestock. Where a non-human mammal is treated or diagnosed, the Bsh3polypeptide, nucleic acid, or antibody employed is preferably specific for that species.

V. Use of Bsh3 Modulators in Tissue and Organ Transplantation

Embodiments of the invention include methods of inhibiting or preventing tissue or organ transplant rejection in a subject, comprising the local administration of a Bsh3 polypeptide, Bsh3 polypeptide fragment, a mitosis-activating peptidomimetic thereof, a transgene encoding a Bsh3 polypeptide, or a transgene encoding a Bsh3 polypeptide fragment to the tissue, organ or to a site proximal to the transplant. Local delivery of the polypeptides or peptidomimetics to the tissue, organ or to a site proximal to the transplant is accomplished by any means commonly available, including but not limited to direct local perfusion, injection, microsponges, microcapsules, liposomes or time-released delivery vehicles.

Local delivery of a transgene encoding a Bsh3 antisense to the tissue, organ or to a site proximal to the transplant may be accomplished with any available vector, via lipofection or via direct plasmid DNA injection. See Qin et al (1995) Transplantation 59(6): 809-816; Le Coultre et al. (1997) Eur. J. Pediatr. Surg. 7(4):221-226; Wang et al. (1992) Transplantation 53(3):703-705; Wang et al. (1996) Transplantation 61(12):1726-1729; Schmid et al., (1997) Eur. J. Cardiothorac. Surg. 11(6):1023-28; and Boasquevisque, C. et al (1997) Ann. Thorac. Surg. 63(6):1556-1561. Vectors encoding the transgene include both replicable and replication-defective vectors, such as retroviral vectors, adenovirus vectors or other vectors with the appropriate tropism for the cells likely to be involved in mitosis or cells proximal to the site of mitosis. In the transgene constructs, expression can be directed from any suitable promoter, including tissue specific promoters which direct gene expression in specific cell types, such as the human insulin promoter. Local delivery of the transgene to the tissue, organ or to a site proximal to the transplant is accomplished by any means commonly available, including but not limited to direct local perfusion, injection, microsponges, microcapsules, liposomes or time-released delivery vehicles.

Without further description, it is believed that one of ordinary skill in the art can, using the preceding description and the following illustrative examples, make and utilize the compounds and practice the claimed methods. The following working examples therefore, specifically point out preferred embodiments of the present invention, and are not to be construed as limiting in any way the remainder of the disclosure. Other generic configurations will be apparent to one skilled in the art. All journal articles and other published documents such as patents and patent applications are hereby incorporated by reference in their entireties.

### EXAMPLES

In Examples 1-3, Bsh3 was identified as a suppressor of the bir1p mutation, bir-46. Thus, the Bsh3 protein can rescue the bir1 mutation and can thus rescue mitosis in these mutated yeast cells.;;

### Example 1

### Materials and Methods

Yeast Strains and Genetic Methods

All S. pombe strains in this study were derived from the wild-type (WT) strain 972 (h⁻) and 975 (h⁺). Standard genetic techniques and media have been described (Guthrie and Fink 1991 Methods Enzymol. 194:1-933, Moreno, et al. 1991 Methods Enzymol. 194: 795-823, and Alfa, et al. 1993, Experiments with fission yeast: a laboratory course manual. Cold Spring Harbor Laboratory Press., Cold Spring Harbor, N.Y.

Generation of bir 1 Deletion Strain

The WT bir1⁺ cDNA was amplified from a cDNA library using standard PCR (Sambrook, et al. 1989 Molecular cloning: a laboratory manual, Cold Spring Harbor Laboratory, Cold Spring Harbor, N.Y,) and subcloned into the plasmid pREP3X, which contains the nmt1 promoter. To generate the S. pombe strain which carried the bir1 deletion in the chromosome but was kept alive by ectopically expressed Bir1p, a WT diploid strain was deleted for one copy of bir1⁺ by one step disruption with his3⁺ (Spd109, h⁻-h⁺ ura4-D18/ura4-D18 leu1-32/leu1-32 hi3-D1/hi3-D1 ade6-M216/ade6-M210 bir1⁺/bir1Δ::his3⁺). The deletion was confirmed by two independent genomic PCR analyses, and tetrad analysis of Spd109 showed 2:2 segregation of 2 viable spores to 2 inviable spores. Spd109 was transformed with pREP3X-bir1⁺ followed by random spore analysis (RSA) and replica-plating.

Spores that were both His⁺ and Leu⁺, indicative of bir1Δ and nmt1-bir1⁺, were selected to generate the controllable Bir1p expression strain (Sp157, h⁻ ura4-D18 leu1-32 his3-D1 ade6-M210 bir1Δ::his3⁺ pREP3X-bir1⁺). The Sp157 strain is very sensitive to thiamine, which represses Bir1p expression from the nmt1 promoter.

To generate the temperature sensitive (ts) allele of bir1, WT genomic bir1⁺ DNA was amplified from genomic DNA using PCR and then subcloned into the plasmid pUR19, which contains the autonomous replication sequence (ARS). The pUR19-bir1⁺ plasmids were subjected to hydroxylamine mutagenesis. Briefly, 10 μg of plasmid was incubated in 500 μl of mutagenesis buffer (1 M hydroxylamine, 50 mM sodium pyrophosphate pH=7.0, 2 mM EDTA and 100 mM NaCl) at 75° C. for 6 to 10 min. Mutagenized plasmids were desalted by QIAEX II gel extraction kit (QIAGEN) and transformed into Sp157 followed by replica-plating. Transformants that were His⁺, Ura⁺, Leu⁻ and ts⁻, indicative of the bir1Δ kept alive by ectopically expressed ts bir1, were selected. To confirm that the ts phenotype did not result from spontaneous mutation, plasmids were isolated and re-transformed into Sp157 followed by replica-plating. One ts allele, bir1-46, was generated. To replace the endogenous bir1with bir1-46, a linear DNA fragment containing ts bir1-46 was transformed into Sp157. Transformants were plated on YES plates to repress Bir1p expression from the nmt1 promoter and enrich for cells with bir1Δ replaced by bir1-46. Transformants were replica-plated and His⁻ and Leu⁻ colonies were selected to generate a ts strain (Sp287, h⁻ bir1-46 his3-D1 ade6-M216 ura4-D18 leu1-32).

To conduct the high-copy suppressor screen, Sp287 (bir-46) was transformed with either a cDNA or genomic DNA library. Transformants were incubated at 28° C. for 20 hr followed by further incubation at 34° C. for five days. For the co-loss analysis, colonies grown at 34° C. were streaked out on YES plates and cultured at 28° C. for 4-5 days to cure the plasmids containing library DNA, followed by replica-plating and incubation at 34° C. to confirm that growth at restrictive temperature is dependent on the library DNA.

The cDNA and genomic DNA of Bsh3⁺ (SEQ ID NOS: 5 and 6 are just the coding sequence) were amplified from WT genomic DNA as Bsh3⁺ has no introns. The Bsh3⁺ cDNA was subcloned into pSGP572 to generate the Bsh3p-GFP fusion construct. To generate the S. pombe strain which carries the Bsh3 deletion in the chromosome but is kept alive by ectopically expressed Bsh3p-GFP, a WT diploid strain was deleted for one copy of Bsh3⁺ by one step disruption with his3⁺ (Spd476, h⁻/h⁺ ura4-D18/ura4-D18 leu1-32/leu1-32 hi3-D1/hi3-D1 ade6-M216/ade6-M210 Bsh3⁺/Bsh3 Δ::his3⁺). Spd476 was transformed with pSGP572-Bsh3⁺-GFP followed by random spore analysis (RSA) and replica-plating. Spores that were both His⁺and Ura⁺, indicative of Bsh3 Δ and nmt1-Bsh3⁺-GFP, were selected to generate the Bsh3p-GFP expression strain (Sp500, h⁻ ura4-D18 leu1-32 his3-D1 ade6-M210 Bsh3 Δ::his3⁺ pSGP572-Bsh3⁺-GFP).

The pic1Δ strain was also generated by one step disruption with his3⁺ (Spd483, h⁻/h⁺ ura4-D18/ura4-D18 leu1-32/leu1-32 hi3-D1/hi3-D1 ade6-M216/ade6-M210 pic1⁺/pic1Δ::his3⁺).

Spore Germination Assay and Yeast Microscopy

For spore germination, diploid cells were cultured in 10 ml YES at 32° C. to late log phase (O.D.₅₉₅=0.8-1.0). Cells were spun down and the pellet was resuspended into 200 ml of ME media followed by 3 days incubation at 25° C. for sporulation. Spores were spun down and resuspended in 20 ml of 2.5% glusalase solution followed by incubation at 28° C. for 24 hr. Glusalase treated spores were washed 5 times with rinse buffer (0.17% yeast nitrogen base) followed by centrifugation through 40 ml of 25% glycerol. Purified spores were cultured in synthetic media without histidine for 24 hr and subjected to DAPI staining.

Yeast immunofluorescence staining was conducted as previously described (Hagan and Hyams 1988, J. Cell. Sci. 89:343-357) except for using zymolyase 100T to digest cell walls (0.5 mg/ml for 7 min at room temperature). Cells were incubated with anti-α-tubulin antibody overnight at room temperature (clone 4A1, 1:100 dilution, kindly provided by Drs. R Macintosh and M. Fuller) followed by 3 washes with PEMBAL buffer (100 mM PIPES pH 6.9, 1 mM EDTA, 1 mM MgSO₄, 1% BSA, 0.1% NaN₃ and 100 mM lysine). Cells were incubated with Texas Red conjugated anti-mouse IgG antibody (1:250 dilution, Southern Biotechnology Associates), for 3-4 hr at room temperature. Cells were washed 3 times with PEMBAL and then subjected to DAPI staining. Cells were viewed using a fluorescence microscope (Olympus 1X70), and images were acquired and deconvolved using DeltVision software (Applied Precision).

### Example 2

### Bir1p is Required for Mitotic Spindle and DNA Damage Response

To study bir1⁺, a S. pombe strain that had a chromosomal bir1 deletion but was kept alive by ectopically expressed Bir1p was generated. Expression of ectopic plasmid Bir1p was controlled by the nmt1 promoter, which was repressed in the presence of thiamine.

Briefly, to identify the phenotypes of the temperature sensitive allele, bir1-46, Wild-type and bir1-46 (Sp287) were streaked out on YES plates or 10 μg/ml of thiabendazole (TBZ), followed by incubation at either 28° C. or 32° C. for 3 days. Wild-type, bir1-46, and a rad3Δ were grown in YES medium to mid log phase (O.D.₅₉₅=0.5-0.6) and then plated on YES plates followed by UV irradiation. Plates were cultured at 28° C. with a foil cover for 1 day and without a foil cover for subsequent 4 days. Colony numbers were calculated and survival rates were determined based on control plates, which were not irradiated. bir1-46 was transformed with vector alone (pUR19), pUR19-bir1⁺, pUR19-Bsh3⁺, or pUR19-pic1⁺. Transformants were streaked out on YES plates followed by incubation at either 28° C. or 34° C. for 3 days. To conduct the high-copy suppressor screen, Sp287 (bir-46) was transformed with either a cDNA or genomic DNA library. Transformants were incubated at 28° C. for 20 hr followed by further incubation at 34° C. for five days.

Consistent with previous results, depletion of Bir1p caused a cut (cellular untimely torn) phenotype in repressing media. Overexpression of human Survivin or cIAP2 failed to complement the viability of bir1Δ. To characterize Bir1p function further, a temperature sensitive (ts) mutant allele, bir1-46, was generated by hydroxylamine mutagenesis and plasmid shuffling (see methods). bir1-46 cells showed the cut phenotype and failed to grow at 32° C., which is the optimal temperature for wild-type fission yeast. In addition, bir-46 also exhibited a slow-growth phenotype at the permissive temperature.

Given that bir1⁺ is required for chromosome segregation and cytokinesis, it is plausible that bir1⁺ is also involved in mitotic spindle integrity. To test this possibility, bir1-46 was challenged with an anti-microtubule drug, Thiabendazole (TBZ). bir1-46 failed to grow in the presence of TBZ at the permissive temperature, suggesting that bir1⁺ plays a role in kinetochore assembly or kinetochore-mitotic spindle attachment. Furthermore, the involvement of bir1⁺ in the DNA damage response to UV irradiation was tested. bir1-46 showed a significant increase in UV sensitivity with increasing dosages of UV radiation, suggesting that bir1⁺ also plays a role in DNA damage response.

### Example 3

### High-Copy Suppressor Screen for bir1-46

To identify genes functioning either downstream or in parallel to bir1⁺, a high-copy suppressor screen was performed. Briefly, Sp287 (bir-46) was transformed with either a cDNA or genomic DNA library. Transformants were incubated at 28° C. for 20 hr followed by further incubation at 34° C. for five days. For the co-loss analysis, colonies grown at 34° C. were streaked out on YES plates and cultured at 28° C. for 4-5 days to cure the plasmids containing library DNA, followed by replica-plating and incubation at 34° C. to confirm that growth at restrictive temperature is dependent on the library DNA.

In this way, suppressors were identified that could complement the growth defect of bir1-46 at the restrictive temperature (34° C.). These suppressors are referred to as bsh (bir1-46 suppressor high-copy). A genomic library DNA fragment was identified that could restore the growth of bir1-46 cells at 34° C. This DNA fragment contained three complete coding regions, arg2⁺, ura5⁺, and a novel gene. Arg2p and Ura5p are involved in arginine and uracil synthesis, respectively. Deletion of the novel gene from the library DNA fragment abolished complementation of the bir1-46 growth defect at 34° C., indicating that this novel gene was a high-copy suppressor for bir1-46.

This novel yeast gene was referred to as Bsh3⁺. Bsh3⁺ encoded a 183 amino acid protein without recognizable protein motifs (SEQ ID NOS: 5 and 6). It shared 29.5% identity with the human homolog, DC5/CGI-122/HSPC037 (SEQ ID NOS:1 and 2), which is referred to as “HBsh3” or “human Bsh3”. In addition to the human homolog, Bsh3p had similar-sized homologs in other species, including M. musculus (AK014776), S. cerevisiae (YJL072c or NP_{—}012463)), Xenopus laevis (AB097169) and C. elegans (F31C3.5), suggesting that Bsh3p is an evolutionarily conserved protein.

### Example 4

### Bsh3p is an Essential Nuclear Protein Required for Cell Growth and Chromosome Segregation

In an attempt to characterize Bsh3⁺, Bsh3⁺ was tested to determine if it was essential for viability in S. pombe using a one-step disruption method and tetrad dissection. A wild-type diploid strain was deleted for one copy of Bsh3⁺ by insertion of the wild-type his3⁺ gene followed by tetrad analysis. The deletion was confirmed by genomic PCR analyses. Briefly, S. pombe Bsh3⁺ was characterized as follows: Bsh3⁺ was shown to be essential when wild-type diploid S. pombe cells (Spd476) deleted for one copy of Bsh3 were subjected to sporulation followed by tetrad dissection. Spores were grown on YES plates at 28° C. for 5 days. The deletion phenotypes of Bsh3 were characterized as follows: Spd476 cells were subjected to large scale sporulation. After glusalase treatment, spores were germinated in synthetic media without histidine for 24 hr followed by DAPI staining. Cells were viewed with a Leitz Laborlux microscope using the 100× objective lens. Images were acquired with Adobe Photoshop software using the SPOT-2 CCD digital camera.

Depletion of Bsh3p resulted in lagging chromosomes. This was shown as follows: Sp571 cells were not depleted or depleted for Bsh3p for 24 hr in repressing medium followed by DAPI staining, or anti-α-tubulin antibody staining and DAPI staining. As a control, Sp571 cells were cultured in non-repressing medium for 24 hr followed by DAPI staining, or anti-α-tubulin antibody staining and DAPI staining. Cells were viewed and images were acquired as described above. The Bsh3⁺/Bsh3 Δ heterozygous diploid had higher rate of chromosome loss than the Bsh3⁺/Bsh3⁺ homozygous diploid when the following test was performed: Bsh3⁺/Bsh3 Δ and Bsh3⁺/Bsh3⁺ cells were cultured in YES medium for 20 hr and then plated on YES plates containing Phloxin B (5 μg/ml). Haploid and diploid cells were determined by the colony color to calculate the haploidization rate. Results of two independent experiments were presented. Cellular localization of Bsh3p was determined as follows: the Bsh3 Δ, nmt1-Bsh3⁺-GFP strain (Sp500) was grown to early log phase (O.D.₅₉₅=0.2) followed by immuno-fluorescence staining analysis (Hagan and Hyams 1988, J. Cell. Sci. 89:343-357). Cells were stained with anti-α-tubulin antibody (clone 4A1) overnight followed by anti-mouse IgG antibody for 3-4 hr (Texas Red conjugated). Cells were then stained with DAPI for 10 min and viewed with an Olympus microscope. Images were acquired and deconvolved using the DeltaVision restoration microscopy system.

Some tetrads showed phenotypic segregation of 2 viable spores to 2 inviable spores, and the rest of the tetrads showed only one viable spore. Furthermore, all viable spores were His, indicating that Bsh3⁺ is essential for cell viability.

To examine the phenotypes of Bsh3 Δ, a spore germination assay was conducted. In short, the Bsh3⁺/Bsh3 Δ::his3⁺ diploid cells were sporulated and treated with glusalase. Spores were germinated in synthetic media without histidine followed by DAPI staining. Deletion of Bsh3⁺ resulted in cell elongation, abnormal and fragmented nuclei, cut and multiple-septation phenotypes. In addition to the spore germination assay, a Bsh3 Δ strain (Sp500) was constructed which was kept alive by a Bsh3p-GFP fusion chimera, ectopically expressed from the nmt1 promoter. In the presence of thiamine, expression of Bsh3p-GFP fusion protein was repressed and cells failed to grow. Consistent with the spore germination result, depletion of Bsh3p for 36 hours at 32° C. caused severe cell elongation with abnormal or fragmented nuclei (30%), modest cell elongation with abnormal or fragmented nuclei (25%), cut (5%) and multiple-septation (17%) phenotypes. The remaining 23% of cells did not show any significant defects in cell morphology or nuclear structure. To further analyze the depletion phenotypes of Bsh3p, a Ndc80p-GFP expressing strain (Sp571, GFP endogenously C-terminally tagged at the ndc80⁺ locus) was used in a Bsh3 Δ genetic background, but kept alive by a nmt1-Bsh3p-myc fusion chimera. Ndc80p is a kinetochore subunit (Wigge and Kilmartin, 2001, J. Cell Biol 152:349-360) and associates with separating chromosomes. However, when Bsh3p was depleted, lagging chromosomes were observed with associated Ndc80p, indicative of chromosome missegregation. In addition, cell elongation was observed with an elongating mitotic spindle and lagging chromosomes in depleted cells. Whereas, complete chromosome segregation and normal nuclear structure were observed when cells were cultured in the non-depleting medium. These observations taken together suggest that Bsh3p is required for proper kinetochore-mitotic spindle attachment.

Since Bsh3p appears to play a role in kinetochore-mitotic spindle attachment, decreased Bsh3p expression should result in chromosome loss due to chromosome missegregation. To test this hypothesis, haploidization assays were used, using Bsh3⁺/Bsh3 Δ heterozygous diploid cells. Previously, it has been shown that diploid S. pombe cells turn into haploid cells when chromosome loss or non-disjunction occurs (Bodi, et al. 1991. Mol Gen Genet 229:77-80, and Snaith, et al. 2000, Mol Cell Biol 20:7922-7932.). A Bsh3 Δ strain (kept alive by nmt1-Bsh3p-myc expression) was crossed to a mat2-B102 strain to generate a stable diploid strain (Spd574, h⁻/h⁺ ura4-D18/ura4-D18 leu1-32/leu1-32 hi3-D1/his3⁺ ade6-M216/ade6-M210 mat2-B102/mat2⁺ Bsh3⁺/Bsh3 Δ), which could not sporulate. Spd574 cells were cultured on 5-FOA containing plates to cure the plasmid which ectopically expresses the Bsh3p-myc fusion protein. Cured cells were cultured in rich medium (YES) for 20 hr and then plated on YES plates containing Phloxin B, a red dye accumulating in diploid cells, to determine the ratio of haploid cells to diploid cells. As a control, a Bsh3⁺/Bsh3⁺ homozygous diploid (Spd573, h⁻/h⁺ ura4-D18/ura4-D18 leu1-32/leu1-32 hi3-D1/his3⁺ ade6-M216/ade6-M210 mat2-B102/mat2⁺ Bsh3⁺/Bsh3⁺) was generated in cells with a mat2-B102 background. More than 97% of Bsh3⁺/Bsh3 Δ heterozygous diploid cells were found to become haploid. In contrast, a very small fraction (2-6.1%) of Bsh3⁺/Bsh3⁺ homozygous diploid cells became haploid, suggesting that decreased expression of Bsh3p leads to a much higher chromosome loss rate resulting from chromosome missegregation.

### Example 5

### Human Bsh3 Localizes in the Nucleus and to the Midbody

Given that Bsh3⁺ plays an essential role in fission yeast, the human homolog, Bsh3, was investigated to determine if it performed a conserved function in vivo. To address this possibility, a Bsh3-GFP (Green Fluorescent Protein) chimera was developed to investigate the cellular localization of human Bsh3. Briefly, cDNA and genomic DNA of Bsh3⁺ were amplified from WT genomic DNA as Bsh3⁺ has no introns. The Bsh3⁺ cDNA was subcloned into pSGP572 to generate the Bsh3p-EGFP fusion construct. To generate the S. pombe strain which carries the Bsh3 deletion in the chromosome but is kept alive by ectopically expressed Bsh3p-GFP, a WT diploid strain was deleted for one copy of Bsh3⁺ by one step disruption with his3⁺ (Spd476, h⁻/h⁺ ura4-D18/ura4-D18 leu1-32/leu1-32 hi3-D1/hi3-D1 ade6-M216/ade6-M210 Bsh3⁺/Bsh3 Δ::his3⁺).

Spd476 was transformed with pSGP572-Bsh3⁺-GFP followed by random spore analysis (RSA) and replica-plating. Spores that were both His⁺ and Ura⁺, indicative of Bsh3 Δ and nmt1-Bsh3⁺-GFP, were selected to generate the Bsh3p-GFP expression strain (Sp500, h⁻ ura4-D18 leu1-32 his3-D1 ade6-M210 Bsh3 Δ::his3⁺ pSGP572-Bsh3⁺-GFP). The full-length human Bsh3 cDNA was amplified from a HeLa cDNA library (Hannon et al. 1993, Genes Dev. 7:2378-2391) using standard PCR and subcloned into the plasmid pEGFP-N1 (Clontech) to generate the Bsh3-EGFP fusion construct. HeLa cells cultured in Dulbecco's Modification of Eagle's Medium (DMEM) containing 10% fetal bovine serum (FBS) were grown in 6-well plates with glass cover slips to ˜30% confluence. Cells were transfected with the Bsh3-EGFP fusion construct using Effectene reagent (QIAGEN) according to the manufacturer's protocol (1:25 ratio). Twenty four hours after transfection, cells were fixed, permeabilized and blocked as previously described (Jiang et al. J. Cell Sci. 114: 3779-3788). The cover slips were incubated with an anti-α-tubulin antibody for 1 hour at room temperature (1:2000 dilution, clone B-5-1-2, Sigma). After 4 washes with PBS, cover slips were incubated with Texas Red conjugated anti-mouse IgG antibody for 1 hour at room temperature (1:250 dilution, Southern Biotechnology Associates). After 3 washes with PBS, cover slips were incubated with PBS containing Hoechst (0.5 mg/ml) for 10-15 min at room temperature. Cells were viewed using a fluorescence microscope (Olympus 1X70), and images were acquired and deconvolved using DeltaVision software (Applied Precision).

The Bsh3-EGFP chimera was ectopically expressed in HeLa cells by transient transfection. Twenty four hours after transfection, localization of the Bsh3-GFP fusion protein was determined by fluorescence microscopy after formaldehyde-sucrose fixation.

Nuclei and mitotic spindles were localized by Hoechst and anti-α-tubulin staining. Ectopically expressed Bsh3-EGFP was found to be localized predominantly in nuclei during interphase and cytokinesis, suggesting that human Bsh3 is also a nuclear protein. The Bsh3-EGFP fusion protein was localized to the midbody during cytokinesis. Furthermore, some Bsh3-EGFP fluorescence was found to associate with the mitotic spindle during metaphase. Thus, human Bsh3-EGFP and S. pombe Bsh3p-GFP showed similar localizations, suggesting that the Bsh3 homologs play a conserved role during mitosis. However, overexpression of human Bsh3 from the nmt1 promoter failed to complement the growth defect of Bsh3 Δ, even though human Bsh3 localized in the nuclei of S. pombe cells.

To investigate the cellular localization of Bsh3p, Sp500 was cultured in thiamine-free media to derepress Bsh3p-GFP expression. The localization of the Bsh3p-GFP fusion protein was determined by fluorescence microscopy after formaldehyde fixation. Nuclei and mitotic spindles were localized by DAPI and anti-α-tubulin staining, respectively. The Bsh3p-GFP fusion protein was localized predominantly in nuclei during both interphase and mitosis, suggesting that Bsh3p is a nuclear protein. Furthermore, the Bsh3p-GFP fusion protein also associated with the elongating mitotic spindle during anaphase, and localized at the central region of the mitotic spindle during telophase.

### Example 6

### Depletion of Human Bsh3 Causes Cell Growth Arrest and Micronuclei Formation, and Chromosome Missegregation

Given that Bsh3p was found to play an essential role in fission of yeast, the human homolog was investigated to determine if it performed a similar function in vivo. To address this possibility, small interfering RNA (siRNA) was used to deplete human Bsh3 in HeLa cells. Since siRNA has been shown to silence gene expression specifically and efficiently in HeLa cells, this method was used to effectively delete Bsh3. The siRNA was designed to match the Bsh3 coding region sequence (nucleotides 111 to 129), and transfected into HeLa cells using Oligofectamine (GibcoBRL). To verify that Bsh3 was depleted, total RNA was isolated three days after transfection followed by RT-PCR (reverse transcription-polymerase chain reaction).

For the siRNA experiments, RNA oligonucleotides—(SEQ ID NO:3) sense CCCUGGUUUACCCGUGGAA-dTdT; antisense: UUCCACGGGUAAACCAGGG-dTdT (SEQ ID NO:4), Dharmacon Research, were deprotected and annealed according to the manufacturer's protocol. siRNA transfection was performed as previously described (Elbashir, et al. 2001, Nature 411:494-498) with minor modifications.

Briefly, HeLa cells cultured with antibiotic-free DMEM and 10% FBS were seeded in 12-well plates (15,000 cells in 1 ml media per well). For each well, 6 μl of 20 μM annealed siRNA was mixed with 100 μl of OptiMem I (GibcoBRL/Invitrogen). In a separate tube, 6 μl of Oligofectamine (GibcoBRL/Invitrogen) was mixed with 24 μl of OptiMem I. After 7 min incubation at room temperature, the two solutions were combined and mixed gently by inversion, followed by further incubation for 20 min at room temperature. 64 μl of OptiMem I was then added to bring up the final volume to 200 μl. 200 μl of the siRNA-Oligofectamine mixture was added to cultured cells. Transfection efficiency was between 80% and 90%, as determined by FITC-conjugated DNA oligonucleotides (21mer).

Three days after transfection, total RNA was isolated using the TRIZOL reagent according to the manufacturer's protocol (GibcoBRL/Invitrogen). Reverse transcription (RT) was conducted in a 10 μl reaction, using 0.5 μg of total RNA and Superscript II reverse transcriptase (GibcoBRL/Invitrogen) according to the manufacturer's protocol. After reverse transcription, 1.5 μl of RT reaction mixture was subjected to PCR (0.2 mM dNTP, 0.1 μM of forward and reverse primers) using Taq DNA polymerase (QIAGEN). The PCR solution was incubated at 93° C. for 3 min, followed by 30 cycles of amplification (92° C. for 35 sec, 52° C. for 40 sec and 72° C. for 1 min). Forward and reverse primers were follows: CGT TGT GGA TCC GAC GCT GCC GAG GTC GAA TTC CTC GCC GAG (forward primer, SEQ ID NO:7) and CTC TAG TCT AGA CTA GAA GTC CTG AGA CTG AGT ACT CTC CAG (reverse primer, SEQ ID NO:8).

Briefly, siRNA analysis of human Bsh3 was as follows: HeLa cells were transfected with either siRNA or buffer alone as a control (Elbashir, et al. 2001, Nature 411:494-498). Three days after transfection, total RNA samples were isolated and subjected to agarose gel (1%) electrophoresis, followed by ethidium bromide staining. Lane 1 had 0.5 μg of siRNA-treated total RNA and lane 2 had 0.5 μg of buffer treated total RNA. Total RNA samples (0.5 μg) were subjected to reverse transcription (RT), followed by PCR (30 cycles) amplification using HBsh3 DNA oligonucleotides and SURVIVIN DNA oligonucleotides as a control. PCR products were subjected to agarose gel (0.7%) electrophoresis followed by ethidium bromide staining.

Lanes 1 and 2 had full-length HBsh3 amplified from total RNA samples treated by siRNA and buffer, respectively; lanes 3 and 4 had full-length SURVIVIN amplified from total RNA samples treated by siRNA and buffer, respectively. Total protein samples (30 μg) were subjected to immuno-blotting analysis using anti-α-tubulin and affinity-purified anti-HsBsh3 antibodies. Lane 1 had siRNA treated cell lysate, lane 2 had buffer treated cell lysate. 100 hr after transfection, cells were fixed with cold 70% ethanol, resuspended in PBS, and stained with propidium iodide (40 μg/ml). Flow cytometry was performed with a Becton Dickinson FACScan and data were analyzed using the CellQuest software. 100 hr after transfection, cells grown on glass cover slips were subjected to Hoechst staining, and images were acquired and deconvolved as described in Example 5.

Total RNA samples isolated from siRNA treated cells and control cells (transfected with buffer alone) showed similar quality and concentration. However, full-length HBsh3 could be amplified from the control total RNA but not the siRNA-treated total RNA. As an internal control, full-length SURVIVIN was isolated from both total RNA samples. Similar amounts of full-length SURVIVIN were amplified from both control total RNA and siRNA-treated total RNA, showing that HBsh3 is specifically depleted by the siRNA. Furthermore, an immunoblotting analysis was performed to confirm that the protein level HBsh3 was also reduced. The anti-α-tubulin antibody detected comparable amounts of α-tubulin in both siRNA and buffer treated cell lysates. However, HBsh3 was detected in the control cell lysate but not the siRNA treated cell lysate, indicating that the Bsh3 protein was depleted.

Interestingly, at 100 hr after transfection, siRNA-treated cells had increased only 5-6 fold in number (2-3 generations), while buffer treated cells increased 20-22 times (>4 generations), suggesting that depletion of HBsh3 results in cell growth arrest. To analyze the phenotype of the siRNA-treated cells further, Fluorescence-activated cell sorting (FACS) analysis was performed. Buffer treated cells had a normal cell cycle profile in which a majority of cells had a 2N DNA content, indicative of G1 phase. siRNA-treated cells had a similar profile except for a significant increase in cells with sub-G1 DNA content (10.1%).

Furthermore, 63% of siRNA treated cells had abnormal and fragmented nuclei (micronuclei), while more than 90% of buffer-treated cells had oval shaped and normal nuclei. Micronuclei and irregular nuclei commonly result from destabilization of the mitotic spindle, or defects in kinetochore-mitotic spindle attachments and/or cytokinesis, as observed in cells lacking Survivin, INCENP, CenpA, CenpC and Bub3. Furthermore, lagging chromosomes were observed in siRNA treated cells during cytokinesis at 68-72 hr after transfection, suggesting that human HBsh3 also plays a role in the kinetochore-mitotic spindle attachment. Our observations taken together suggest that depletion of Bsh3 arrests cell growth and results in sub-G1 DNA content and irregular nuclei, due to chromosome missegregation or incomplete nuclear fusion caused by cytokinesis defects.

To further analyze the mitotic defects apparent in Bsh3-depleted cells, siRNA depletion was performed using thymidine-synchronized cells. Briefly, HeLa cells were blocked by thymidine 40 hr after siRNA transfection. After 20 hr of thymidine treatment, cells were washed and cultured in thymidine free media for 14 hr, followed by immunofluorescence staining analysis using the anti-centromere antibody, ANA-C (Sigma). Chromosomes and mitotic spindles were localized by Hoechst and anti-α-tubulin staining, respectively. Most buffer-transfected metaphase cells (95%) showed normally aligned and congressed chromosomes. In contrast, 40% of siRNA-transfected metaphase cells showed unaligned chromosomes at the spindle pole region, suggesting that depletion of Bsh3 causes either premature chromosome segregation or defective chromosome congression. To distinguish these two possibilities, siRNA depletion and video-microscopy were performed using a HeLa cell line expressing the histone H2B-GFP fusion protein. While the wild-type cells were able to congress and segregate their chromosomes in 45-60 min, siRNA treated cells (30%) took more than 2 hr for the same process due to the fact that some chromosomes were delayed in alignment at the metaphase plate. Consistent with this result, 14 hr after thymidine block and release, 3.2% of siRNA-treated cells remained in metaphase (Table 1). However, only 0.34% of buffer-treated cells remained in metaphase (Table 1). Taken together these observations suggest that human Bsh3 is required for proper chromosome congression. This observation also suggests that depletion of human Bsh3 might result in unattached or mono-oriented chromosomes, which are much more difficult to align at the metaphase plate.

TABLE 1
Comparison of sensitivities of buffer- and Bsh3
siRNA- treated cells to Vinblastine (VBL)

| Transfection | % of cells in metaphase | | | |
|---|---|---|---|---|
| (VBL conc.) | No VBL control | 1 nM | 0.5 nM | 0.1 nM |
| Buffer | 0.3% | 13.6% | 1.7% | 1.5% |
| Bsh3 siRNA | 3.2% | 36.2% | 23.6% | 10.3% |

Vinblastine Sensitivity

To confirm this interpretation, it was analyzed whether human Bsh3 depleted cells were more sensitive to Vinblastine (VBL), a microtubule-disrupting drug. It has been shown that a low concentration of VBL (nM) alters microtubule dynamics without depolymerizing microtubules. As a result, it affects chromosome movement during metaphase and delays chromosome congression. In addition Vinblastine disrupts kinetochore tension without affecting spindle-kinetochore attachment.

siRNA depletion using thymidine-synchronized cells, which were released into culture media with different concentrations of VBL for 14 hr, was followed by immunofluorescence staining as described in the previous section. In the presence of low amounts of VBL (Table 1, 0.5 and 0.1 nM), siRNA-treated cells exhibited a much higher metaphase population (more than 10-fold) than control cells, suggesting that human Bsh3-depleted cells are much more sensitive to low concentrations of VBL. This result shows that human Bsh3 plays a role in spindle microtubule-kinetochore attachment and/or microtubule dynamics, which are required for chromosome movement and alignment during metaphase.

Immunofluorescence Staining for Mitotic Proteins

To investigate whether depletion of human Bsh3 affects cellular localization of other proteins involved in chromosome congression, such as kinetochore proteins and chromosomal passenger proteins, immunofluorescence staining was performed using human Bsh3-depleted HeLa cells. Survivin, Hec1, CENP-E, Prc1, Aurora-B kinase and BUBR1 (checkpoint protein) were identified. However, depletion of human Bsh3 using siRNA did not affect the localization of Survivin, Hec1, CENP-E, Prc1 or Aurora-B significantly. Interestingly, the kinetochore staining intensity of BUBR1 associated with unaligned chromosomes was 3.9 fold higher than the low kinetochore staining intensity of chromosomes aligned at the metaphase plate (pixel intensity was measured using the DeltaVision software). This observation is consistent with previous reports that kinetochores of unaligned chromosomes, such as prometaphase chromosomes or VBL-treated metaphase chromosomes, show higher BUBR1 staining intensity.

Measurement of the Distance Between Kinetochores

In addition, the distance between sister kinetochores was measured to address whether depletion of human Bsh3 affects tension between paired kinetochores. Immunofluorescence staining was performed using anti-CENP-E antibody and human Bsh3-depleted HeLa cells. The distance between sister kinetochores was measured by the DeltaVision software. As a control, we treated cells with nocodazole to determine the distance between sister kinetochores without tension. We found that the distance between paired kinetochores was shorter in siRNA-transfected cells than buffer-transfected cells during prometaphase and under nocodazole treatment. During metaphase, there was no significant difference in the distance between paired kinetochores aligned at the metaphase plate in siRNA and buffer-transfected cells. However, the distance between sister kinetochores of unaligned chromosomes in siRNA transfected cells was much shorter, consistent with reduced tension. Our data taken together suggest that depletion of human Bsh3 causes unstable kinetochore-spindle attachment and/or decreased tension between kinetochore pairs, which is normally generated by microtubule dynamics during chromosome congression.

### Example 7

### Pic1p (S. pombe Inner Centromere Protein) Functions as a High-Copy Suppressor of bir1-46

In addition to the high-copy suppressor screen, reversion analysis was performed to identify extragenic suppressors of the TBZ sensitivity of bir1-46. These suppressors were referred to as bst (bir1-46 suppressors on TBZ plates). Two suppressors, bst1 and bst2, were isolated which showed temperature sensitivity and the cut phenotype at 34° C. (Huang and Hunter, unpublished data). While trying to clone bst1⁺ and bst2⁺, overexpression of Bsh3⁺ or pic1⁺ (SPBC336.15-SPBC685.01) was found to rescue the growth defects of bst1 and bst2 (Huang and Hunter, unpublished data). The inner centromere protein (INCENP), a chromosomal passenger protein, is required for chromosome segregation and cytokinesis. Given that overexpression of Bsh3⁺ rescued the growth defect of bir1-46 at 34° C., pic1⁺ was tested to see whether it also functions as a high-copy suppressor of bir1-46. Overexpression of pic1⁺ complemented the growth defect of bir1-46 at 34° C. (FIG. 1C), indicating that pic1⁺ is also a high-copy suppressor of bir1-46.

### Example 8

### Bsh3 is Involved in Bir1p Localization and Translocation from Chromosome to Mitotic Spindle

Since Bsh3p functions as a high-copy suppressor for bir1-46, it was possible that Bir1p mislocalization might underlie the chromosome segregation defects in Bsh3p depleted cells. The localization of Bir1p was determined by fluorescence microscopy using affinity purified anti-Bir1p antibodies.

Briefly, polyclonal anti-serum against human Bsh3 was raised in rabbits using His-tagged full-length human Bsh3 as the antigen. Anti-Bsh3 antibodies were purified using affinity purification with minor modifications (Harlow and Lane, 1988 Antibodies, a laboratory manual. Cold Spring Harbor Laboratory, Cold Spring Harbor, N.Y.). In short, the GST-HsBsh3 fusion proteins (full-length) were purified and immobilized on glutathione agarose beads using dimethylpimelidate (5 mg/ml). Poly-clonal anti-serum was diluted 1 in 6 in PBS and passed through HsBsh3-beads, followed by washes using PBS and PBS with 500 mM NaCl. Bound antibodies were eluted with 0.2 M ethanolamine (pH 11) and 0.1 M glycine (pH 2.9).

Nuclei and mitotic spindles were localized by DAPI and anti-α-tubulin antibody staining, respectively. Bir1p is known to localize predominantly in nuclei during interphase, and concentrates at the outer centromere region, appearing as a discrete spot inside the nucleus during metaphase. During anaphase Bir1p associates with the elongating mitotic spindle and then localizes to the central region of the mitotic spindle during late anaphase and telophase.

To test whether Bsh3p depletion affected Bir1p localization Sp499 cells were switched to repressive medium to shut off Bsh3p expression. Because Bsh3p appears to be a stable protein and because it is overepxressed, complete depletion takes more than 30 hr (i.e. several generations). At 30 hr after Bsh3p depletion, chromosome segregation appeared to be normal. Bir1p associated with the mitotic spindle during early anaphase (98% of anaphase cells), and localized in part to the central region of the elongated spindle in telophase, as expected. In contrast, at 36 hr after Bsh3p depletion, when no Bsh3p-myc protein was detectable, nuclei divided abnormally during early anaphase, and late anaphase. Moreover, Bir1p localization showed two different defects. First, Bir1p remained associated with DNA in a bright discrete spot (76% of anaphase cells), which resembled the staining pattern seen during metaphase. These results suggest that Bsh3p might be required for translocation of Bir1p from chromosomes to the mitotic spindle. Second, as shown in Table 3, 22% of anaphase cells showed either diffuse or no distinct Bir1p staining. Similar defects in Bir1p localization were also observed in Bsh3p-depleted late anaphase cells, where 54% of telophase cells showed abnormal or no appreciable Bir1p staining. Deletion of Bsh3 abolished proper localization of Birp1. These results suggest that Bsh3p is required for proper Bir1p localization. In contrast, Bsh3p depletion did not affect the localization of Swi6p, a heterochromatin protein, or Ndc80p, an outer kinetochore protein.

TABLE 2
Bsh3p depletion causes severe defects in Bir1p localization

| Mitotic phase | | | |
|---|---|---|---|
| (Bir1p localization) | % of cells showing abnormal localization | | |
| (depletion time) | 30 hr | 33 hr | 36 hr |
| Anaphase (spindle) | 0% | 0% | 22% |
| Telophase (central spindle) | 6% | 6% | 54% |
| (for each timepoint 100 cells | | | |
| were counted | | | |

### Inhibitors of Bsh3 and pic1+ as Antifungals

From the data above, it can be seen that in addition to anti-tumor uses, antisense and inhibitors of Bsh3 and pic1+ may be used as antifungals. For example, they may be used to treat any disease caused by yeast or other fungi in humans or animals, including but not limited to: thrush, yeast infections, and systemic infections. Alternatively, they may be used to treat diseases caused by yeast in plants.

While this invention has been described with respect to various specific examples and embodiments, it is to be understood that the invention is not limited thereto and that it can be variously practiced within the scope of the following claims.

What is claimed is:

1. A method for identifying a compound that modulates Bsh3 levels in a cell, comprising:
a) contacting the cell with a test compound; and
b) monitoring the level of Bsh3 in the cell.

2. The method of claim 1, wherein said Bsh3 has the amino acid sequence of SEQ ID NO: 2.

3. The method of claim 1, wherein said test compound is an inhibitor of Bsh3 protein activity.

4. The method of claim 3, wherein said inhibitor is an siRNA molecule.

5. The method of claim 1, wherein said test compound is an activator of Bsh3 protein activity.

6. The method of claim 1, wherein said test compound associates indirectly with said Bsh3 protein.

7. The method of claim 1, wherein said test compound affects transcription or translation of a Bsh3 gene encoding said Bsh3 protein.

8. The method of claim 7, wherein said Bsh3 gene has the nucleotide sequence of SEQ ID NO: 1.

9. The method of claim 1, wherein said test compound binds to said Bsh3 protein.

10. A method for modulating the growth of cells in vitro, comprising:
contacting said cells with an effective amount of a compound that modulates Bsh3 protein activity in said cells; and
monitoring the growth of said cells.

11. The method of claim 10, wherein said Bsh3 protein has the amino acid sequence of SEQ ID NO: 2.

12. The method of claim 10, wherein said compound is an inhibitor of Bsh3 protein activity and said modulated growth is reduced growth.

13. The method of claim 10, wherein said compound is an activator of Bsh3 protein activity and said modulated growth is increased growth.

14. The method of claim 10, wherein said compound affects transcription or translation of a Bsh3 gene encoding said Bsh3 protein.

15. The method of claim 14, wherein said Bsh3 gene has the nucleotide sequence of SEQ ID NO: 1.

16. A method for inhibiting the growth of cells, comprising contacting said cells with an effective amount of a compound that reduces Bsh3 protein activity in said cells.

17. The method of claim 16, wherein the cells are selected from the group consisting of: cancer cells, tumor cells, autoimmune reactive cells and fungal cells.

18. The method of claim 16, wherein said compound is an siRNA molecule.

19. The method of claim 16, wherein said contacting is in vitro.

20. A method of lowering the effective dose of an antineoplastic agent on cancer cells, comprising contacting said cancer cells with a compound that reduces the Bsh3 protein activity in said cancer cells.

21. The method of claim 20, wherein said antineoplastic agent is selected from the group consisting of: colchicine, vinblastine and vincristine.

22. The method of claim 20, wherein said cancer cells are tumor cells.

23. The method of claim 20, wherein said compound is an siRNA molecule.

24. The method of claim 20, wherein said Bsh3 protein has the amino acid sequence of SEQ ID NO: 2.

25. A method for increasing mitosis in a population of cells, comprising contacting said cells with an effective amount of Bsh3 protein.

26. A method of monitoring the proliferation potential of a population of cells comprising determining the level of BSH3 activity or expression in the cell population.

27. A method of monitoring the effectiveness of a cancer therapy in a patient comprising:
providing a patient undergoing therapy to reduce a population of cancer cells; and
determining the level of Bsh3 activity or expression in the population of cancer cells following the treatment, wherein a decreased level of Bsh3 is indicative of an effective therapy.
